(19) United States
(12) Patent Application Publication
(10) Pub. No.: US 2021/0186965 A1
(43) Pub. Date: Jun. 24, 2021

(54) COMPOSITION COMPRISING TWO ENZYME INHIBITORS TARGETING TWO DIFFERENT CONFORMATIONS OF AN ENZYME

(71) Applicant: University College Dublin, Dublin (IE)

(72) Inventors: Oleksii Rukhlenko, Dublin (IE); Jens Rauch, Dublin (IE); Walter Kolch, Dublin (IE); Boris Kholodenko, Dublin (IE)

(21) Appl. No.: 17/057,384

(22) PCT Filed: May 21, 2019
(86) PCT No.: PCT/EP2019/063153

§ 371 Date: Nov. 20, 2020

(30) Foreign Application Priority Data
May 21, 2018 (GB) 1808321.2

(51) Int. Cl. A61K 31/506; A61P 35/00; A61K 31/437; A61K 45/06; A61K 31/4409

(52) U.S. Cl. CPC A61K 31/506; A61P 35/00; A61K 31/4409; A61K 45/06; A61K 31/437

(57) ABSTRACT

The present invention composition relates to compositions and combination therapies for use in the prevention, management, amelioration or treatment of a cancer or RASopathy disorders in which targeted therapy is used, the composition comprising two enzyme inhibitors targeting two different conformations of an enzyme, or enzymes in the same functional family, implicated in the cancer or RASopathy disorders. The present invention also relates to methods for identifying enzyme inhibitors which may be suitable for use in treatments.

### TECHNICAL FIELD OF THE INVENTION

The present invention relates to compositions and combinations comprising two enzyme inhibitors targeting two different conformations of an enzyme, or enzymes in the same functional family, implicated in cancer or RASopathies.

### BACKGROUND TO THE INVENTION

The RAS/RAF/MEK/ERK pathway is pivotal for cell proliferation and survival and is frequently hyperactivated in tumors. Oncogenic mutations in the RAS genes (H-RAS, K-RAS, and N-RAS) occur in about 30% of cancers (Prior et al., 2012; Stephen et al.). In addition to cancer, germline mutations in genes encoding MAPK pathway components are associated with a group of developmental disorders known as RASopathies or RAS/MAPK syndromes (Rauch et al., 2016). Biochemical studies of these mutants as well as structural analysis and network-level data suggest that MAPK pathway activation in RASopathies is quantitatively, rather than qualitatively, different compared to cancer-related mutations (Rauch et al., 2016). Despite a three-decade long effort at developing RAS inhibitors, there is still no clinically available drug. As a result, the development of inhibitors of the kinases downstream of RAS has become a hot topic in drug development (Caunt et al., 2015; Rahman et al., 2014). Considerable efforts have focused on RAF kinases, owing to frequent BRAF mutations that drive cancer and developmental disorders (Rauch et al., 2016). The most common oncogenic BRAF mutation, BRAFV600E is found in ca 8% of human tumors and 60% of melanomas (Weinstein et al., 2013; Holderfield et al., 2014)). The ATP-competitive RAF inhibitors in clinical use, vemurafenib and dabrafenib, show high initial response rates in patients with mutant BRAFV600E malignant melanomas, but the effects are short-lived (Holderfield et al., 2014). Moreover, about 30% of patients develop secondary skin hypertrophy or malignances because of paradoxical ERK activation in wild-type (WT) BRAF cells (Yaktapour et al., 2014). Paradoxical ERK activation is particularly pronounced in mutant RAS tumors conveying intrinsic resistance to RAF inhibitors (Zhang et al., 2015), which can even accelerate tumor growth and invasion (Sanchez-Laorden et al., 2014).

Homo- and hetero-dimerization of the RAF kinases ARAF, BRAF and CRAF (gene name RAF1) significantly increases their catalytic activities and represents a key event in the activation of normal and oncogenic RAF pathways (Freeman et al., 2013; Garnett et al., 2005; Rushworth et al., 2006). The binding of RAF molecules to active RAS drives RAF dimerization by inducing conformational changes, dephosphorylation of inhibitory residues and bringing RAF molecules into proximity of each other (Dhillon et al., 2002; Kholodenko et al., 2000; Weber et al., 2001). Enhanced RAF kinase dimerization driven by oncogenic RAS mutations or upregulation of upstream receptors leads to intrinsic or acquired resistance to RAF inhibitors (Lito et al., 2013; Nazarian et al., 2010). Other resistance mechanisms connected with increased RAF dimerization include CRAF overexpression (Holderfield et al., 2014; Lito et al., 2013), BRAF amplification (Shi et al., 2012), and BRAFV600E splice variants exhibiting enhanced dimerization potential (Poulikakos et al., 2011). All clinically used RAF inhibitors are ineffective against RAS mutant tumors (Hatzivassiliou et al., 2010; Poulikakos et al., 2010) and show poor performance in BRAF mutant colorectal cancers (Holderfield et al., 2014). Thus, more effective therapeutic strategies are currently needed to target mutant BRAF driven cancers.

Protein kinases toggle between inactive and active conformations that differ by the positions of the highly conserved DFG motif and αC-helix. ATP-competitive RAF inhibitors can be classified based on their preferential binding to different (IN or OUT) conformations of the DFG motif and αC-helix (IN and OUT positions correspond to active and inactive kinase conformations, respectively) (Fabbro, 2015; Karoulia et al., 2016; Roskoski, 2016). A broad classification includes three inhibitor types: αC-IN/DFG-IN (denoted CI/DI, Type I), αC-OUT/DFG-IN (CO/DI, Type I 1/2), and αC-IN/DFG-OUT (CI/DO, Type II), see Table 1 below. The observation that ATP-competitive inhibitors bind with different affinities to active and inactive kinase conformations received much attention in the drug discovery effort, but mostly in terms of inhibitor structures. The inventors have recently reported that fundamental thermodynamic principles governing allosteric inhibitor effects can explain both paradoxical RAF kinase activation and common resistance mechanisms to RAF inhibitors (Kholodenko, 2015). This work suggested that a combination of two structurally different RAF inhibitors may offer a path to abolish resistance (Kholodenko, 2015). However, to understand which inhibitor types to combine and in which cellular contexts, we need to connect thermodynamic and structural analyses of inhibitor-RAF interactions with biochemical, mutational and pathway regulation data, including dynamics of posttranslational modifications (PTMs) and feedback loops.

Intrinsic or acquired resistance to kinase inhibitors, including RAF inhibitors in melanoma and other cancers remains a pressing clinical problem. While different combinations of kinase inhibitors are routinely tested in clinical trials, it is unclear how the best combinations can be chosen. A plethora of confounding factors, including allosteric drug-kinase interactions, phosphorylation-induced conformational changes and kinase dimerization, multiple feedback loops and diverse cell mutational and expression profiles hamper intuitive reasoning about optimal drug combinations. Understanding each drug's mode of action and the mode of their combined actions at the network level would enable a systematic and robust design of the best combinations. Different dynamics of phosphorylation responses to inhibitors that preferentially bind to active or inactive conformations have been previously reported (Kleiman et al., 2011).

Clinically used RAF inhibitors are ineffective in RAS-mutant tumors, enhancing homo- and heterodimerization of RAF kinases, and leading to paradoxical activation of ERK signaling (Karoulia et al., 2016). Numerous mechanisms of RAF inhibitor resistance result in enhanced RAF dimerization and cannot be overcome by existing RAF inhibitors.

It is an object of the present invention to address one or more of the above problems associated with kinase inhibitors. It is an object of the present invention to provide for a therapy which can be used to address intrinsic or acquired resistance to kinase inhibitors. It is an object of the present invention to provide for a therapy which can be used to address RAS-mutant tumors.

### SUMMARY OF THE INVENTION

In accordance with a first aspect of the present invention, there is provided a composition for use in the prevention, management, amelioration or treatment of a cancer, the composition comprising two enzyme inhibitors targeting two different conformations of an enzyme, or enzymes in the same functional family, implicated in the cancer.

The enzyme activation may include kinase dimerization or oligomerization which is a component of the onset or progress of the cancer and the targeting of said enzymes causes inhibition of the enzyme dimers or oligomers. The two synergistic enzyme inhibitors will preferably act synergistically.

In accordance with a related aspect of the present invention, there is provided a composition for use in a method of prevention, management, amelioration or treatment of cancer, the method comprising administering a therapeutically effective amount of the composition to a subject in need thereof, the composition comprising two enzyme inhibitors targeting two different conformations of an enzyme, or enzymes in the same functional family, implicated in cancer.

In accordance with a further related aspect of the present invention, there is provided a composition for use in the manufacture of a medicament for the prevention, management, amelioration or treatment of cancer, the composition comprising two enzyme inhibitors targeting two different conformations of an enzyme, or enzymes in the same functional family, implicated in the cancer.

In accordance with a further related aspect of the present invention, there is provided a composition for use in the prevention, management, amelioration or treatment of a cancer, where the cancer involves enzyme activation which includes homodimerization or heterodimerization of an enzyme and/or within enzymes of the same enzyme family, and where the composition comprises enzyme inhibitors which are capable of changing allosteric interactions of enzyme protomers in a dimer and targeting different conformations of the same enzyme, or enzymes in the same enzyme family.

In accordance with another related aspect of the present invention, there is provided a composition for use in the prevention, management, amelioration or treatment of a RASopathy disorder, the composition comprising two enzyme inhibitors targeting two different conformations of an enzyme, or enzymes in the same functional family, implicated in the RASopathy disorder.

In accordance with a related aspect of the present invention, there is provided a composition for use in a method of prevention, management, amelioration or treatment of a RASopathy disorder, the method comprising administering a therapeutically effective amount of the composition to a subject in need thereof, the composition comprising two enzyme inhibitors targeting two different conformations of an enzyme, or enzymes in the same functional family, implicated in the RASopathy disorder.

In accordance with a further related aspect of the present invention, there is provided a composition for use in the manufacture of a medicament for the prevention, management, amelioration or treatment of a RASopathy disorder, the composition comprising two enzyme inhibitors targeting two different conformations of an enzyme, or enzymes in the same functional family, implicated in the RASopathy disorder.

In accordance with a further related aspect of the present invention, there is provided a composition for use in the prevention, management, amelioration or treatment of a RASopathy disorder, where the RASopathy disorder involves enzyme activation which includes homodimerization or heterodimerization of an enzyme and/or within enzymes of the same enzyme family, and where the composition comprises enzyme inhibitors which are capable of changing allosteric interactions of enzyme protomers in a dimer and targeting different conformations of the same enzyme, or enzymes in the same enzyme family.

Preferably, the enzyme, or enzymes, comprise a kinase or kinases and/or a pseudokinase or pseudokinases, and the two enzyme inhibitors comprise two kinase inhibitors. The two kinase inhibitors may target alternative conformations of the DFG motif and αC-helix on the kinase. The two kinase inhibitors may be of different types. The different types may comprise Type I and Type II or Type I 1/2 and Type II kinase inhibitors. Alternatively, at least one of the kinase inhibitors may comprise a covalent inhibitor.

It will be apparent to the skilled addressee that the two enzyme inhibitors may be selected to targeting particular kinases. Preferably, the two enzyme inhibitors are two RAF family inhibitors, two ErbB family inhibitors or two JAK family inhibitors.

The composition may be used to treat a number of cancers, RASopathies or other disorders. The disease may bear activating mutations and/or overexpressed proteins in the RAS (NRAS, HRAS, KRAS), RAF (BRAF, CRAF, ARAF), ErbB (Her1/EGFR/ErbB1, Her2/Neu/ErbB2, Her3/ErbB3, and Her4/ErbB4) and JAK (JAK1, JAK2, JAK3, and TYK2) family proteins, existing individually or in combinations.

Advantageously, the inventors have identified that each enzyme inhibitor may be present is present in the composition at a lower dose than would typically be used individually to treat the cancer or RASopathy disorder. That is to say that the two enzyme inhibitors have been shown to have a synergistic effect which enables lowered doses of the enzyme inhibitors to be used. It will be apparent that this will reduce toxicity issues experienced by individuals and may also enable treatment of individuals previously excluded from treatment regimens due to sensitivity to toxicity.

In accordance with a second aspect of the present invention, there is provided combination therapy for use in the prevention, management, amelioration or treatment of cancer, the combination comprising two enzyme inhibitors targeting two different conformations of an enzyme, or enzymes in the same functional family, implicated in the cancer.

The enzyme activation may include kinase dimerization or oligomerization which is a component of the onset or progress of the cancer and the targeting of said enzymes causes inhibition of the enzyme dimers or oligomers. The two synergistic enzyme inhibitors will preferably act synergistically.

In a related aspect of the present invention, there is provided a combination therapy for use in a method of prevention, management, amelioration or treatment of cancer, the method comprising administering a therapeutically effective amount of the composition to a subject in need thereof, the combination comprising two enzyme inhibitors targeting two different conformations of an enzyme, or enzymes in the same functional family, implicated in the cancer.

In accordance with a further related aspect of the present invention, there is provided a combination therapy for use in the manufacture of a medicament for the prevention, management, amelioration or treatment of cancer, the combination comprising two enzyme inhibitors targeting two different conformations of an enzyme, or enzymes in the same functional family, implicated in the cancer.

In accordance with a further aspect of the present invention, there is provided a combination therapy for use in the prevention, management, amelioration or treatment of a cancer, where the cancer involves enzyme activation which includes homodimerization or heterodimerization of an enzyme and/or within enzymes of the same enzyme family, and where the combination comprises enzyme inhibitors which are capable of changing allosteric interactions of enzyme protomers in a dimer and targeting different conformations of the same enzyme, or enzymes in the same enzyme family.

In accordance with a further aspect of the present invention, there is provided combination therapy for use in the prevention, management, amelioration or treatment of a RASopathy disorder, the combination comprising two enzyme inhibitors targeting two different conformations of an enzyme, or enzymes in the same functional family, implicated in the RASopathy disorder.

In a related aspect of the present invention, there is provided a combination therapy for use in a method of prevention, management, amelioration or treatment of a RASopathy disorder, the method comprising administering a therapeutically effective amount of the composition to a subject in need thereof, the combination comprising two enzyme inhibitors targeting two different conformations of an enzyme, or enzymes in the same functional family, implicated in the RASopathy disorder.

In accordance with a further related aspect of the present invention, there is provided a combination therapy for use in the manufacture of a medicament for the prevention, management, amelioration or treatment of a RASopathy disorder, the combination comprising two enzyme inhibitors targeting two different conformations of an enzyme, or enzymes in the same functional family, implicated in the RASopathy disorder.

In accordance with a further aspect of the present invention, there is provided a combination therapy for use in the prevention, management, amelioration or treatment of a RASopathy disorder, where the RASopathy disorder involves enzyme activation which includes homodimerization or heterodimerization of an enzyme and/or within enzymes of the same enzyme family, and where the combination comprises enzyme inhibitors which are capable of changing allosteric interactions of enzyme protomers in a dimer and targeting different conformations of the same enzyme, or enzymes in the same enzyme family.

Preferably, the enzyme, or enzymes, comprise a kinase or kinases and the two enzyme inhibitors comprise two kinase inhibitors. The two kinase inhibitors may target alternative conformations of the DFG motif and αC-helix on the kinase. The two kinase inhibitors may be of different types. The different types may comprise Type I and Type II or Type I 1/2 and Type II kinase inhibitors. Alternatively, at least one of the kinase inhibitors may comprise a covalent inhibitor.

It will be apparent to the skilled addressee that the two enzyme inhibitors may be selected to target particular kinases. Preferably, the two enzyme inhibitors are two RAF family inhibitors, two ErbB family inhibitors or two JAK family inhibitors.

The combination may be used to treat a number of cancers or RASopathy disorders. The disease may bear activating mutations and/or overexpressed proteins in the RAS (NRAS, HRAS, KRAS), RAF (BRAF, CRAF, ARAF), ErbB (Her1/EGFR/ErbB1, Her2/Neu/ErbB2, Her3/ErbB3, and Her4/ErbB4) and JAK (JAK1, JAK2, JAK3, and TYK2) family kinases, existing individually or in combinations.

Advantageously, as mentioned earlier, the inventors have identified that each enzyme inhibitor may be present is present in the composition at a lower dose than would typically be used individually to treat the disease.

Each enzyme inhibitor for the above described composition or combination may be selected from one the following groups:

(i) Type I RAF inhibitors (e.g SB-590885, GDC-0879, etc.)

(ii) Type I1/2 RAF inhibitors (e.g. vemurafenib, dabrafenib, LGX818, PLX8394, etc.);

(iii) Type II RAF inhibitors (e.g. sorafenib, AZ-628, TAK-632, LY3009120, BGB283, etc.)

(iv) Type I ErbB inhibitors (e.g. gefitinib, erlotinib, etc.);

(v) Type I1/2 ErbB inhibitors (e.g. lapatinib, etc.)

(vi) Type I JAK inhibitors (e.g. tofacitinib, ruxolitinib, etc.);

(vii) Type II JAK inhibitors (e.g. BBT-594, CHZ868, etc.)

In accordance with a third aspect of the present invention, there is provided a method of identifying two or more compounds for incorporation into a combination therapy for the use in the treatment of cancer or a RASopathy disorder, where enzyme activation including kinase dimerization is a component of the onset or progress of the cancer or RASopathy disorder, the method comprising the steps of:

a) identifying if the cancer or RASopathy disorder involves enzyme activation which includes homodimerization or heterodimerization of the enzyme and/or within the same enzyme family; and

b) identifying if enzyme inhibitors are capable of changing allosteric interactions of enzyme protomers in a dimer; and

c) selecting two inhibitors targeting different conformations of the same enzyme, or enzymes in the same enzyme family, and including the inhibitors in a combination therapy.

In accordance with a fourth aspect of the present invention, there is provided a method of formulating a composition for use in the prevention, management, amelioration or treatment of a cancer or RASopathy disorder, the composition comprising combining two enzyme inhibitors capable of targeting two different conformations of an enzyme, or enzymes in the same functional family, implicated in the cancer or RASopathy disorder.

The enzyme activation may include kinase dimerization or oligomerization which is a component of the onset or progress of the cancer or RASopathy disorder and the targeting of said enzymes causes inhibition of the enzyme dimers or oligomers. The two synergistic enzyme inhibitors will preferably act synergistically.

The method may be used to produce a composition or combination therapy as herein above described. In the method, the cancer or RASopathy disorder may involve enzyme activation including kinase dimerization. Furthermore, the enzyme activation may include homodimerization or heterodimerization with the enzyme, or within the same enzyme family. Preferably, the inhibitors are capable of changing allosteric interactions of enzyme protomers in a dimer.

Advantageously, the inventors have found that by using a combined experimental and computational approach, they could build a mechanistic dynamic model to analyze combinations of structurally different RAF inhibitors, which can efficiently suppress MEK/ERK signaling. This next-generation model of the RAS/ERK pathway integrates thermodynamics and kinetics of drug-protein interactions, structural elements, post-translational modifications and cell mutational status, predicting best RAF inhibitor combinations for cancer cells harboring oncogenic RAS and/or BRAFV600E. Synergistic inhibition of ERK signaling in mutant NRAS, HRAS and BRAFV600E cells was corroborated by experiments, demonstrating the power of structure-based dynamic modeling.

Furthermore, this comprehensive model is based on extended studies of RAF kinase regulation by multiple phosphosites and dimerization, and intensive RAF inhibitor research. The model predicts a number of surprising, and unexpected properties of network responses to different types of RAF inhibitors and makes new strides in understanding resistance to these drugs. The model suggests that synergy can emerge between Type I and Type II, as well as between Type I 1/2 and Type II inhibitors and predicts new ways of overcoming RAF inhibitor resistance in RAS mutant cells. The experimental results on responses of MEK/ERK signaling to different RAF inhibitor types and their combinations in melanoma cells bearing oncogenic RAS, BRAFV600E mutations, or both BRAFV600E and NRAS mutations support model predictions Inhibition of oncogenic RAS signaling in MEL-JUSO cells (NRAS^{Q61L/WT}, HRAS^{G13D/G13D}) is associated with reduced cell proliferation and colony formation. The results suggest a new principle of targeting the same kinase with two structurally different inhibitors that bind to different kinase conformations.

As used herein, the terms “treatment”, “treating”, “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting or slowing its development; and (c) relieving the disease, i.e., causing regression of the disease.

The term “subject” used herein includes any human or nonhuman animal. The term “nonhuman animal” includes all mammals, such as nonhuman primates, sheep, dogs, cats, cows, horses.

A “therapeutically effective amount” refers to the amount of inhibitors that, when administered to a subject for treating a disease, is sufficient to affect such treatment for the disease. The “therapeutically effective amount” will vary depending on the inhibitor(s) used, the disease and its severity and the age, weight, etc., of the subject to be treated.

In general, routes of administration contemplated by the invention include, but are not necessarily limited to, enteral, parenteral, or inhalational routes.

Parenteral routes of administration other than inhalation administration include, but are not necessarily limited to, topical, transdermal, subcutaneous, intramuscular, intraorbital, intracapsular, intraspinal, intrasternal, intrathecal, and intravenous routes, i.e., any route of administration other than through the alimentary canal. Parenteral administration can be carried to effect systemic or local delivery. Where systemic delivery is desired, administration typically involves invasive or systemically absorbed topical or mucosal administration of pharmaceutical preparations. Enteral routes of administration include, but are not necessarily limited to, oral and rectal (e.g., using a suppository) delivery.

Conventional and pharmaceutically acceptable routes of administration include intranasal, intramuscular, intra-tracheal, intrathecal, intracranial, subcutaneous, intradermal, topical, intravenous, intraperitoneal, intra-arterial (for example, via the carotid artery), spinal or brain delivery, rectal, nasal, oral, and other enteral and parenteral routes of administration.

In some embodiments, a composition of the invention, or a combination of the invention, may be administered with one or more other compounds effective for the prevention, management, amelioration or treatment of an age-related disease or condition or cancer.

The inhibitors may be artificially generated. That is to say that it is not naturally occurring. The inhibitors may however be a naturally occurring molecules whose concentration and formulation in a medicament or pharmaceutical preparation or combination enables it to be used for the prevention, management, amelioration or treatment of cancer or RASopathy disorder, whereas otherwise it would have no or limited efficacy. Whilst the inhibitors may be naturally occurring molecules, it will be understood that the concentration and formulation of the molecules found to be therapeutically effective would not be present in nature at such a concentration or in a formulation with other components such as excipients.

One or more of the inhibitors may comprise an antibody or antibodies or antibody mixture. Such antibody or antibodies may be polyclonal or may be monoclonal. It will be apparent to the skilled addressee how to produce antibodies which would act as inhibitors. Preferably the antibodies will be humanised.

In other embodiments, the inhibitor or inhibitors comprise a peptide or peptide mimetic thereof, or C-terminal amidated peptide thereof.

The terms “peptide” and “peptides” include compounds that have amino acid residues (H-Cα-[side chain]) but which may be joined by peptide (—CO—NH—) or non-peptide linkages. Peptides may be synthesised by the Fmoc-polyamide mode of solid-phase peptide synthesis.

The peptide may be a peptide aptamer. Peptide aptamers typically consist of short, 5-20 amino acid residues long sequences that can bind to a specific target molecule.

There are a number of different approaches to the design and synthesis of peptide composition that do not contain amide bonds. In one approach, one or more amide bonds are replaced in an essentially isoteric manner by a variety of chemical functional groups.

Retro-inverso peptidomimetics, in which the peptide bonds are reversed, can be synthesised by methods known in the art. This approach involves making pseudopeptides containing changes involving the backbone, and not the orientation of side chains. Retro-inverse peptides, which contain NH—CO bonds instead of CO—NH peptide bonds, are more resistant to proteolysis.

The peptide may be linear. Although, it may be advantageous to introduce a cyclic moiety into a peptide-based framework. The cyclic moiety restricts the conformational space of the peptide structure and this may lead to an increased efficacy. An added advantage of this strategy is that the introduction of a cyclic moiety into a peptide may also result in the peptide having a diminished sensitivity to cellular peptidases.

In some embodiments of the invention the peptide may be joined to another moiety. Convenient moieties to which the peptide may be joined include polyethylene glycol (PEG) and peptide sequences, such as TAT and antennapedia which enhance delivery to cells.

In some embodiments, the inhibitor or inhibitors is/are pro-drugs of the peptide. A pro-drug is a compound which is metabolised in vivo to produce the molecule, such as a protein. One of skill in the art will be familiar with the preparation of pro-drugs.

The peptide may be a peptide mimetic. A peptide mimetic is an organic compound having similar geometry and polarity to the molecules defined herein, and which has a substantially similar function. A mimetic may be a molecule in which the NH groups of one or more peptide links are replaced by CH₂ groups. A mimetic may be a molecule in which one or more amino acid residues is replaced by an aryl group, such as a napthyl group.

In other embodiments, an inhibitor or inhibitors comprise nucleic acid, such as single stranded DNA or RNA, which is capable of binding to and inhibiting ERK and AKT. It is envisaged that the same targets on ERK and AKT are also suitable for targeting with peptides and peptide aptamers will also be suitable for targeting with RNA or modified RNA aptamers. Nucleic acids such as single stranded DNAs and RNAs may be provided that bind to and inhibit ERK and AKT. Typically, the nucleic acids are single stranded and have from 100 to 5000 bases.

In yet other embodiments, an inhibitor or inhibitors comprise a small molecule or small molecules. The small molecule may be any appropriate organic molecule that inhibits the targeted enzyme, or family group of enzymes.

Features, integers, characteristics, compounds, molecules, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and figures), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### DETAILED DESCRIPTION OF THE INVENTION

Embodiments of the invention are described below, by way of example only, with reference to the accompanying figures in which:

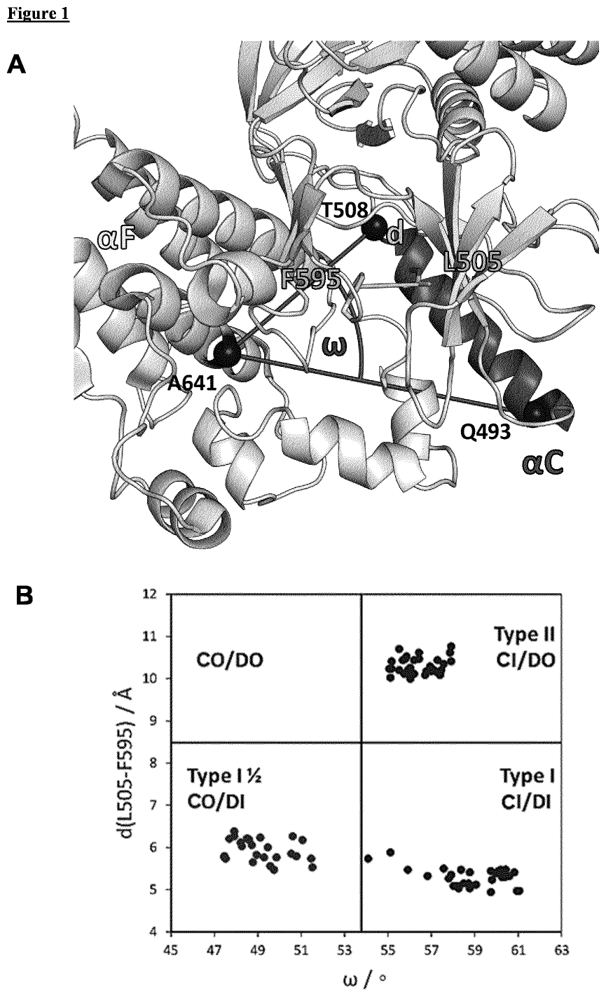

FIG. 1. Asymmetry of BRAF homodimers. (A) Definitions of the αC-helix angle (ω) IN (ω>54°) or OUT (ω<52°) positions (Jambrina et al., 2014) as well as the DFG-motif IN (d<7 Å) or OUT (d>9.5 Å) conformations via the L505-F595 distance (d). The structure is illustrated using the PDB structure 3TV4. (B) Distribution of the αC-helix and DFG motif conformations in the 90 BRAF protomers analyzed. Among 45 analyzed RAF dimer PDB structures, 6 of the structures contain only 1 inhibitor molecule, and the rest have two inhibitors molecules bound to the dimer;

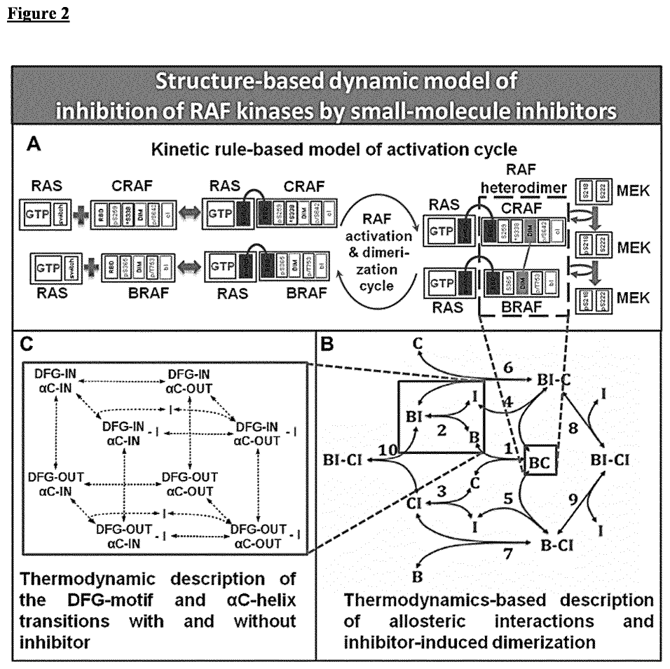

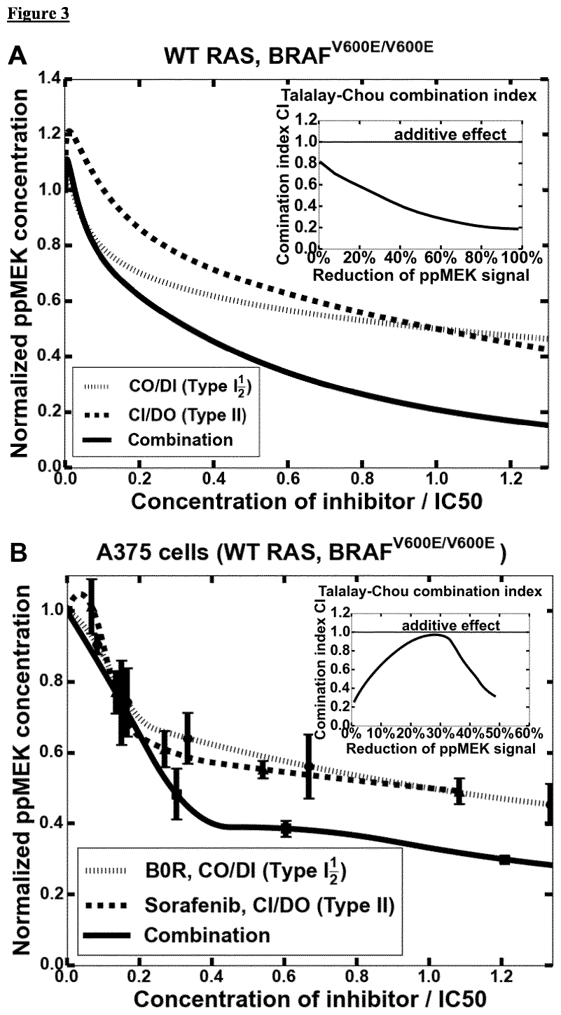

FIG. 2. Rule-based modeling of binding and phosphorylation reactions and concomitant conformational changes of RAF kinases. (A) Illustration of rules governing RAF binding to RAS and RAF activation and dimerization cycles. Protein domains and phosphosites that are phosphorylated (p) or dephosphorylated are shown by rectangles. Switch is the RAS switch domain. RBD and DIM are the RAS-binding and dimerization domains; bI and cI are the inhibitor binding sites on BRAF and CRAF. The domains that are bound in a protein complex are colored. In the S338 rectangle the asterisk (*) indicates that the S338 phosphorylation state does not influence the CRAF association/dissociation reactions with RAS-GTP, whereas the rates of those reactions are affected by the states of the RAF residues (pS642 and pT753) that are phosphorylated by ERK. (B) Simplified BRAF-CRAF (B-C) dimerization cycle and allosteric inhibitor (I) interactions with RAF monomers and dimers. (C) The reaction of inhibitor (I) binding to BRAF (reaction 2) is expanded into 12 reactions that take into account possible positions of the DFG-motif and αC-helix;

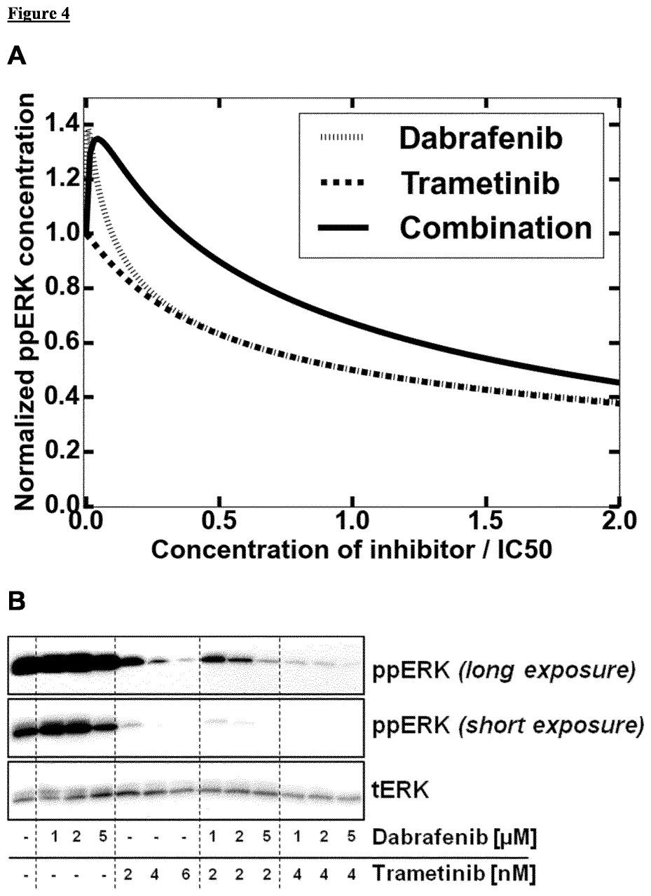

FIG. 3. Combination of CO/DI and CI/DO drugs synergistically inhibits the ERK pathway in cells bearing BRAFV600E mutation and WT RAS. (A) Model-predicted responses of MEK signaling to CO/DI and CI/DO inhibitors and their combinations in cells with homozygous BRAFV600E mutation. [RAS-GTP]=25 nM, [BRAF^{V600E}]=50 nM, [BRAF^{WT}]=0, basal ppMEK level is 609 nM. (B) MEK signaling responses of growing A375 (WT RAS, BRAF^{V600E/V600E}) cells to sorafenib measured using MESOSCALE system, 1 hr, IC50 of sorafenib is 44 μM. [RAS-GTP]=25 nM, [BRAF^{V600E}]=25 nM, [BRAF^{WT}]=25 nM, basal ppERK level is 1363 nM. (A) The ratio of CO/DI and CI/DO inhibitor doses is 1.2:1. The remaining parameters are given in (Rukhlenko et al., 2018). In each panel, the insert assesses drug synergy using the Talalay-Chou combination index (CI);

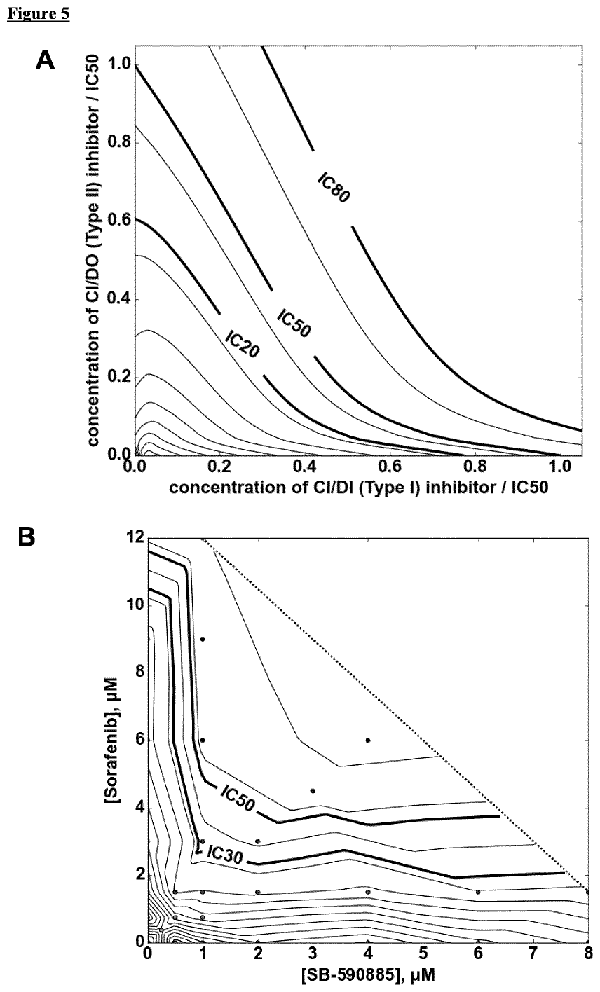

FIG. 4. A combination of dabrafenib and trametinib shows antagonism in cells harboring oncogenic RAS mutation and WT BRAF. (A) Simulated responses of ERK signaling to individual drugs and their combination. Inhibitor doses are normalized by IC50. [RAS-GTP]=250 nM, [BRAF^{WT}]=50 nM, [BRAF^{V600E}]=0, basal ppERK level is 480 nM. The ratio of CO/DI RAF (dabrafenib) and MEK inhibitor (trametinib) normalized doses applied in combination is 1:6.4. (B) ERK signaling responses of growing MEL-JUSO cells (NRAS^{Q61L/QT}, HRAS^{G13D/G13D}, BRAF^{WT/WT}) to dabrafenib (CO/DI RAF inhibitor), trametinib (MEK inhibitor) and their combination measured using Western Blot, 1 hr;

FIG. 5. Combination of CI/DI and CI/DO inhibitors synergistically inhibits the ERK pathway in cells harboring oncogenic RAS mutations and WT BRAF. (A) Simulated stationary responses of ERK signaling to individual drugs and their combinations. Inhibitor doses are normalized by IC50. [RAS-GTP]=250 nM, [BRAF^{V600E}]=0, [BRAF^{WT}]=50 nM, basal ppERK level is 480 nM. (B) ERK signaling responses of growing MEL-JUSO cells (NRAS^{Q61L/QT}, HRAS^{G13D/G13D}, BRAF^{WT/WT}) to SB-590885 (CI/DI), sorafenib (CI/DO) and their combination measured using LUMINEX system, 24 hr. The ppERK responses are plotted vs the absolute concentrations of inhibitors applied separately and vs the sum of absolute concentrations for their combination;

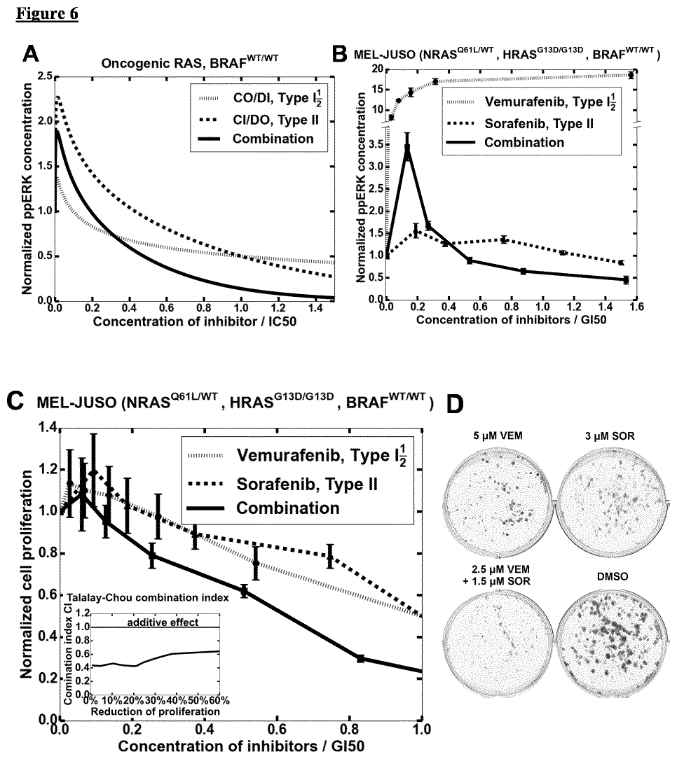

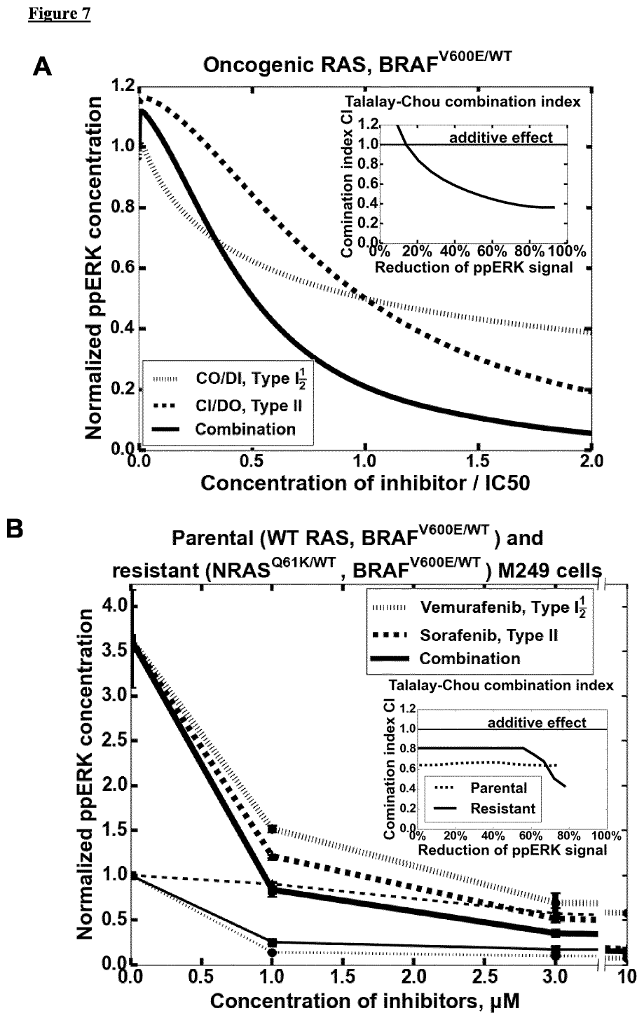

FIG. 6. Combination of CI/DO and CO/DI inhibitors synergistically inhibits ERK signaling, proliferation, and colony formation in cells bearing oncogenic RAS mutations and WT BRAF. (A) Simulated responses of ERK signaling to CO/DI and CI/DO inhibitors and their combination. Inhibitor doses are normalized by IC50, the total doses shown for the combination correspond to the optimal dose ratio that is nearly 1:1. [RAS-GTP]=250 nM, [BRAF^{V600E}]=0, [BRAF^{WT}]=50 nM, basal ppERK level is 484 nM. (B, C) ERK signaling (B) and cell proliferation (C) responses of growing MEL-JUSO cells (NRAS^{Q61L/WT}HRAS^{G13D/G13D}, BRAF^{WT/WT}) to vemurafenib (CO/DI), sorafenib (CI/DO) and their combination measured using MESOSCALE system, 24 hr treatment. Doses are normalized by G150 levels, i.e. by the levels of 50% inhibition of cellular growth. The ratio of vemurafenib and sorafenib applied in combination is 1:1. (C) Error bars are calculated using 4 biological replicates. The Talalay-Chou combination index (CI) assessing drug synergy in inhibiting cell proliferation is shown as insert. (D) Colony formation of MEL-JUSO cells treated with vemurafenib (VEM) and sorafenib (SOR) applied separately and in combination, a representative of 3 biological replicates;

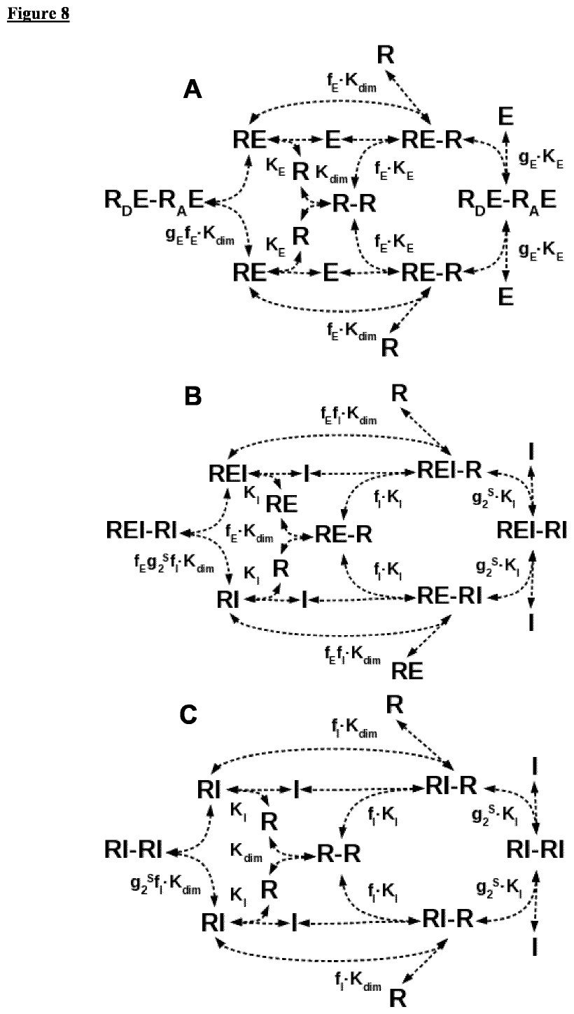

FIG. 7. Combinations of RAF inhibitors can synergistically inhibit ERK signaling in cells bearing both oncogenic RAS and BRAFV600E mutations. (A) Model-predicted stationary responses of ERK signaling to CI/DO and CO/DI inhibitors and their combinations. Inhibitor doses are normalized by IC50. In combinations, the ratio of CO/DI and CI/DO inhibitor doses are 1:1. [RAS-GTP]=250 nM, [BRAF^{V600E}]=25 nM, [BRAF^{WT}]=25 nM, basal ppERK level is 2151 nM. (B) ERK signaling responses to vemurafenib (CO/DI), sorafenib (CI/DO) and a combination measured using MESOSCALE system, 24 hr treatment for growing parental (BRAF^{V600E/WT}/WT RAS) and resistant (BRAF^{V600E/WT}/NRAS^{Q61K/WT}) M249 cells. The ppERK response is plotted vs the absolute concentrations of inhibitors applied separately and vs the sum of absolute concentrations for combinations, in which the ratio of vemurafenib and sorafenib doses is 1:1. Inserts assess drug synergy using the Talalay-Chou combination index (CI);

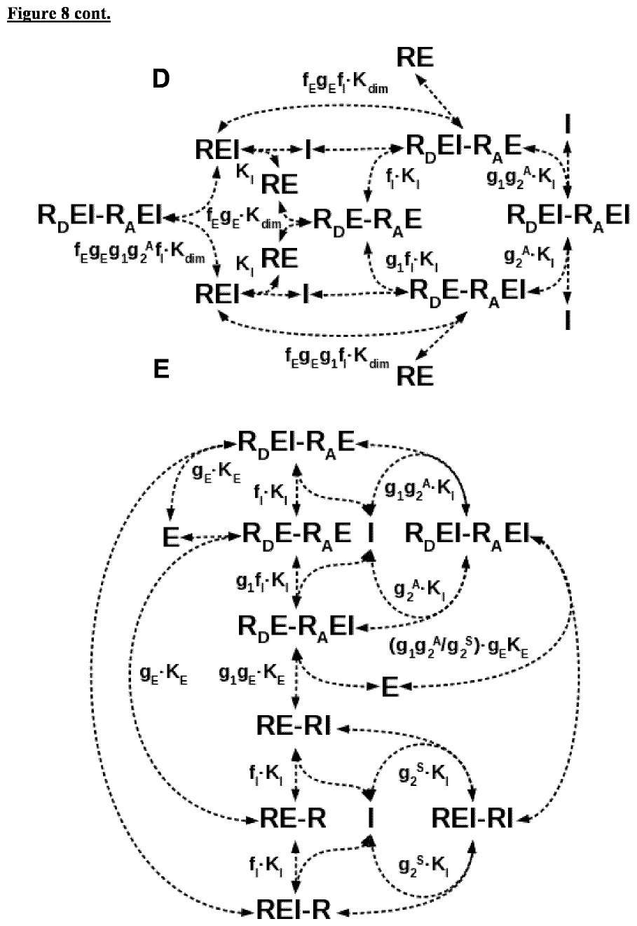

FIG. 8. Kinetic schemes of ErbB dimerization, and ligand and inhibitor binding. (A) Binding of ligands and ErbB dimerization. R, free receptor monomer; E, ligand; R-R are RE-R kinase inactive, symmetric dimers (free or harbouing a single ligand molecule); R_{D}E-R_{A}E, a kinase active, asymmetric dimer. (B) Binding of an inhibitor (I) to a free symmetric ErbB dimer. (C) Binding of an inhibitor to a symmetric ErbB dimer harbouring one ligand molecule. (D) Binding of an inhibitor to an asymmetric ErbB dimer harbouring two ligand molecules. (E) Binding of both a ligand and an inhibitor to a symmetric ErbB dimer, RE-R;

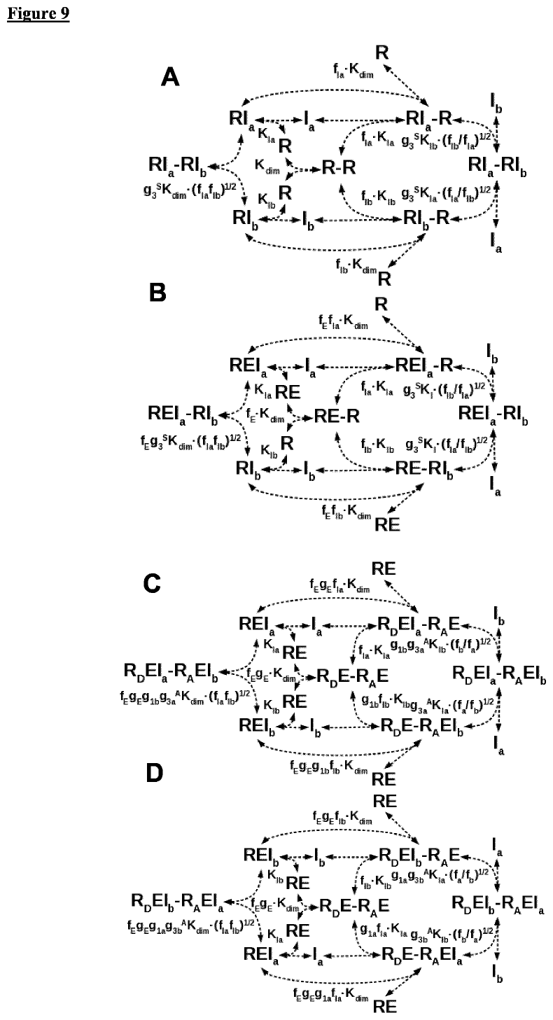

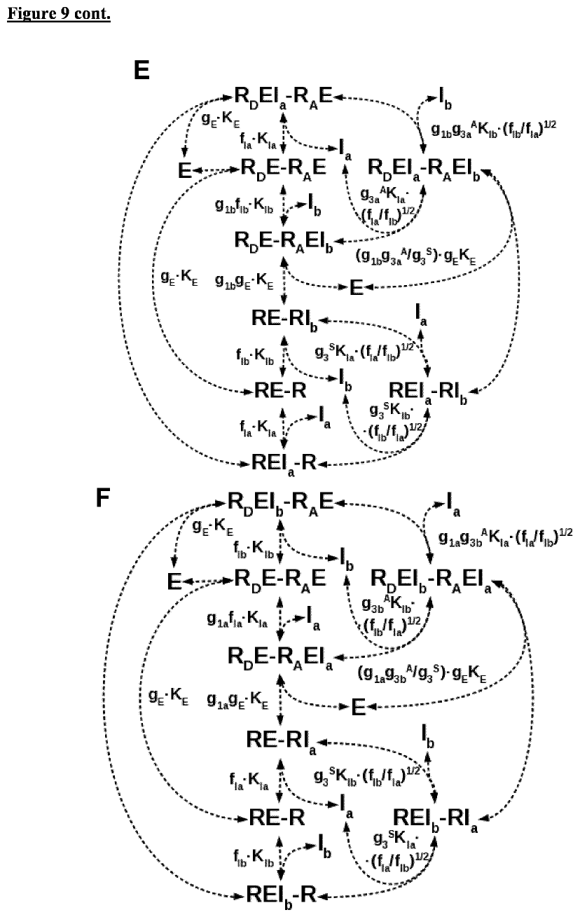

FIG. 9. Kinetic schemes of ErbB dimerization and binding of two different inhibitors. (A) Binding of two inhibitors (Iₐ and I_{b}) to a free symmetric ErbB dimer. R, free receptor monomer; R—R is a free, kinase inactive symmetric dimer (B) Binding of two inhibitors to a symmetric ErbB dimer harbouring one ligand molecule. RE-R is a kinase inactive, symmetric dimer harbouring one molecule of ligand. (C-D) Binding of two inhibitors to an asymmetric ErbB dimer harbouring two ligand molecules. R_{D}E-R_{A}E is a kinase active asymmetric dimer. Relations between the dissociation constants are indicated using the thermodynamic parameters, explained in the text. (E-F) Binding of both a ligand and two inhibitors to a symmetric ErbB dimer, RE-R;

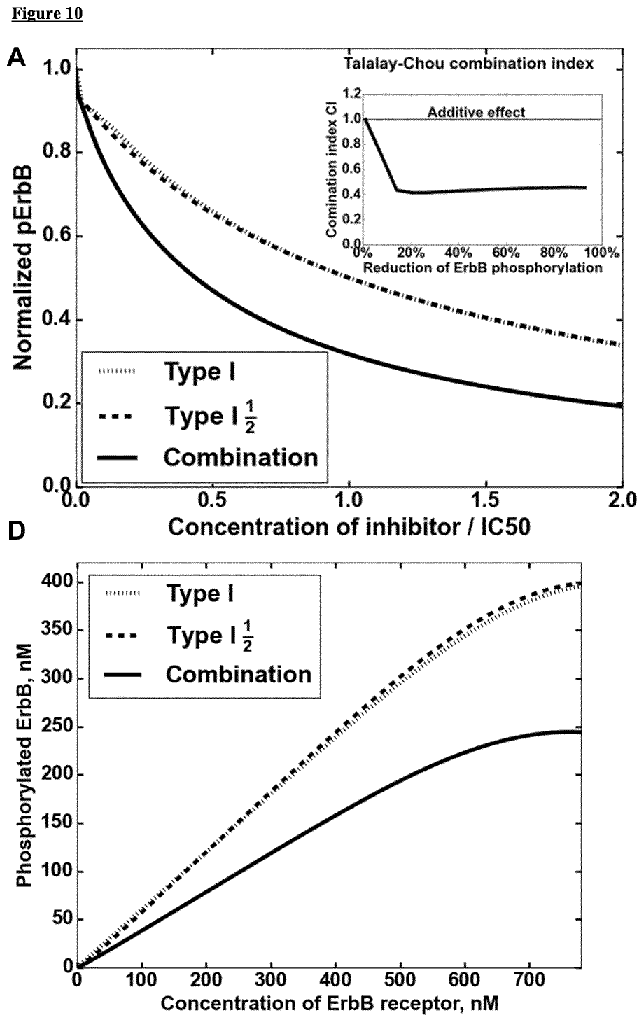

FIG. 10. Structure-based mathematical model predicts synergy between type I and type I1/2 ErbB inhibitors. (A) Model-predicted responses of ErbB phosphorylation to type I and type I1/2 ErbB inhibitors and their combination. Insert shows assessment of synergy between type I and type I1/2 ErbB inhibitors by means of Talalay-Chou combination index, which shows marked synergy for medium and high levels of inhibition of ErbB phosphorylation. (B) Model-predicted dependence of ErbB phosphorylation on total concentration of ErbB receptor for type I and type I1/2 inhibitors used on their own and in combination;

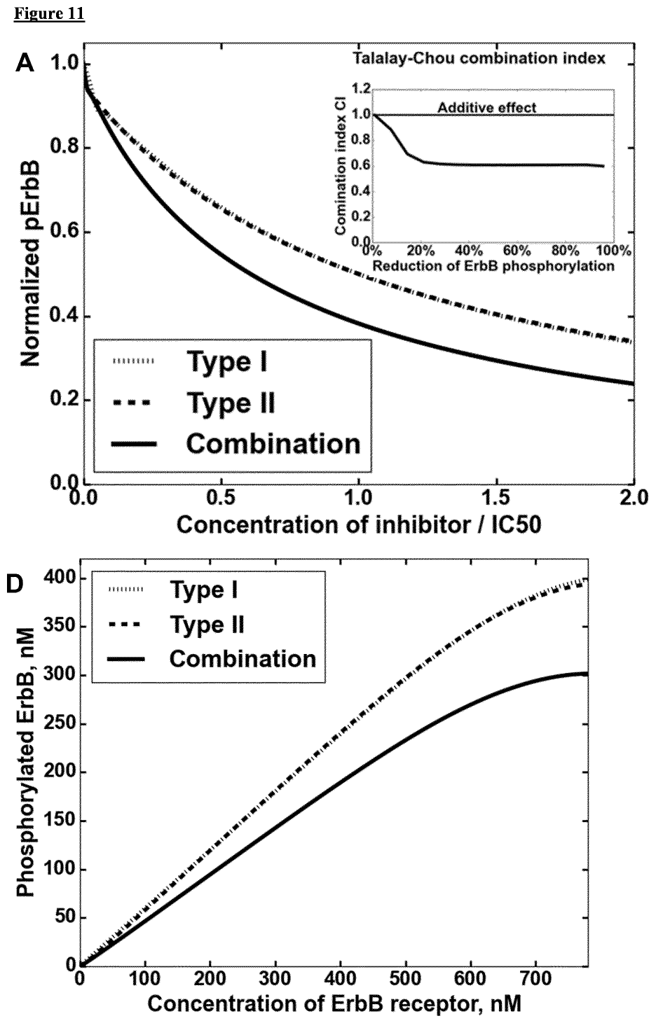

FIG. 11. Structure-based mathematical model predicts synergy between type I and type II ErbB inhibitors. (A) Model-predicted responses of ErbB phosphorylation to type I and type II ErbB inhibitors and their combination. Insert shows assessment of synergy between type I and type I1/2 ErbB inhibitors by means of Talalay-Chou combination index, which shows marked synergy for medium and high levels of inhibition of ErbB phosphorylation. (D) Model-predicted dependence of ErbB phosphorylation on total concentration of ErbB receptor for type I and type II inhibitors used on their own and in combination;

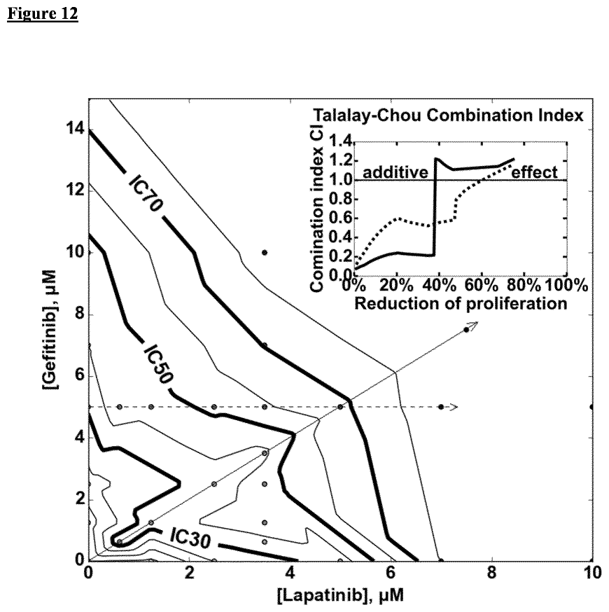

FIG. 12. Combination of Laptinib and Gefitinib Inhibitors Synergistically Inhibits Proliferation of HER2-positive breast cancer cells (SKBR3) Proliferation of SKBR3 (HER2-positive breast cancer) cells in response to Lapatinib (Type I1/2), Gefitinib (Type I) and combination treatment (4 days). Shown is a representative of 3 biological replicates. Black lines represent Loewe isoboles of constant inhibition of cellular proliferation. Inserts represent assessment of synergy strength by means of Talalay-Chou combination index for specific directions shown by arrows. Combination index shows marked synergy for intermediate levels of drugs.

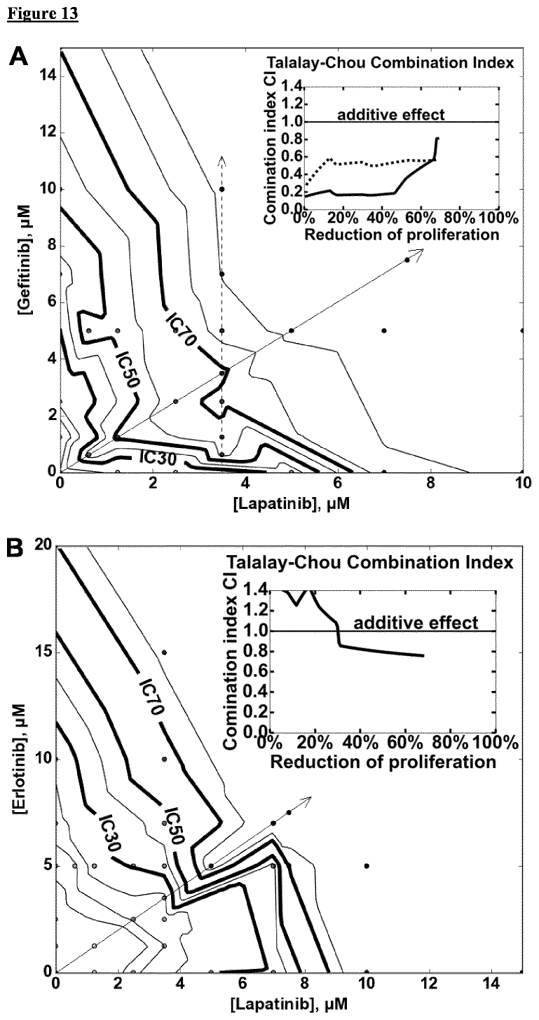

FIG. 13. Combinations of Laptinib/Gefitinib (A) and Laptinib/Erlotinib (B) Inhibitors Synergistically Inhibit Proliferation of HER2-positive breast cancer cells (AU565). Proliferation of AU565 (HER2-positive breast cancer) cells in response to Lapatinib (Type I1/2), Gefitinib (Type I), Erlotinib (Type I) and combination treatment (4 days). Shown is a representative of 3 biological replicates. Black lines represent Loewe isoboles of constant inhibition of cellular proliferation. Inserts represent assessment of synergy strength by means of Talalay-Chou combination index for specific directions shown by arrows. Combination index shows marked synergy for intermediate and high levels of drugs.

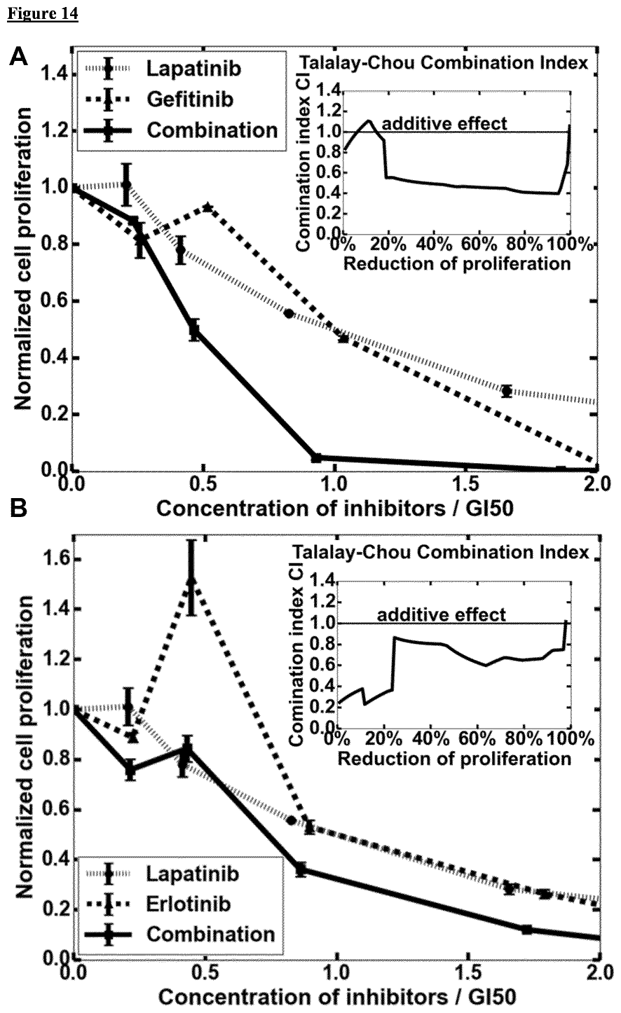

FIG. 14. Combinations of Laptinib/Gefitinib (A) and Laptinib/Erlotinib (B) Inhibitors Synergistically Inhibit Proliferation of HER2-positive breast cancer cells (BT474). Proliferation of BT474 (HER2-positive breast cancer) cells in response to Lapatinib (Type I1/2), Gefitinib (Type I), Erlotinib (Type I) and combination treatment (4 days). Shown are representatives of 3 biological replicates. Error bars indicate standard error of mean. Insert represents assessment of synergy strength by means of Talalay-Chou combination index. Combination index shows marked synergy for intermediate and high levels of drugs.

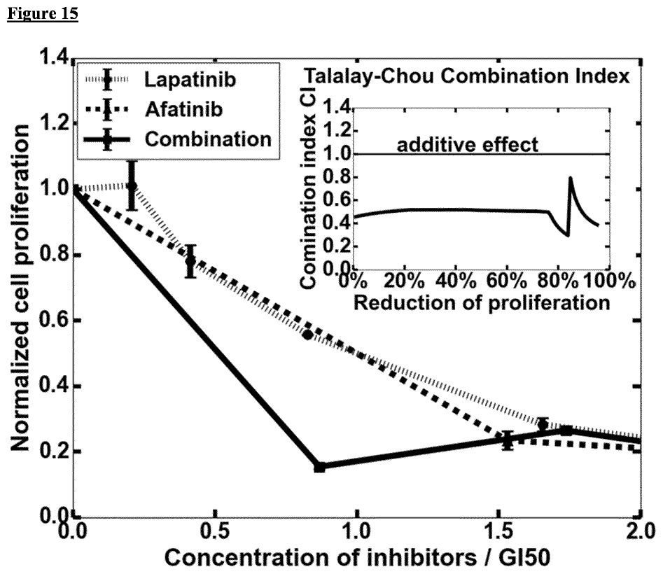

FIG. 15. Combination of Laptinib and Afatinib Inhibitors Synergistically Inhibit Proliferation of HER2-positive breast cancer cells (BT474). Proliferation of BT474 (HER2-positive breast cancer) cells in response to Lapatinib (Type I1/2), Afatinib (covalent inhibitor binding to kinase active conformation) and combination treatment (4 days). Shown is a representative of 3 biological replicates. Error bars indicate standard error of mean. Insert represents assessment of synergy strength by means of Talalay-Chou combination index. Combination index shows marked synergy for all drug levels.

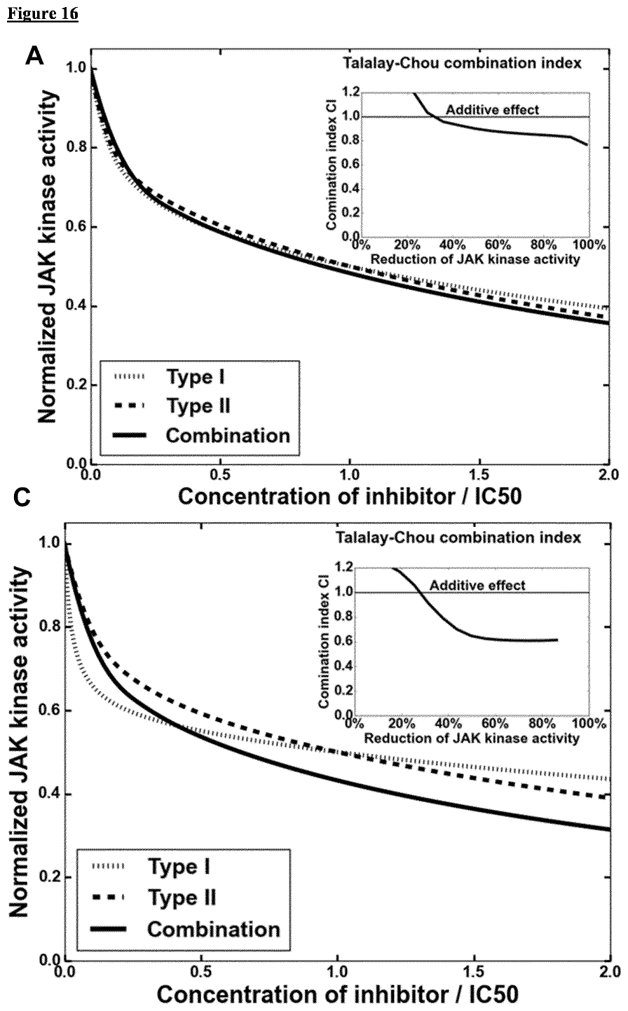

FIG. 16. Numerical simulations suggest that combination of type I and type II JAK inhibitors synergistically inhibits signalling downstream of JAK in cells with asymmetric JAK dimers, while in cells with symmetric JAK dimers the effect of combination is close to additive. Model-calculated responses of JAK kinase activity to type I and type II JAK inhibitors and their combination in cells harbouring symmetric (A) asymmetric (B) JAK dimers. Inserts represent of the synergy strength assessed by the Talalay-Chou combination index.

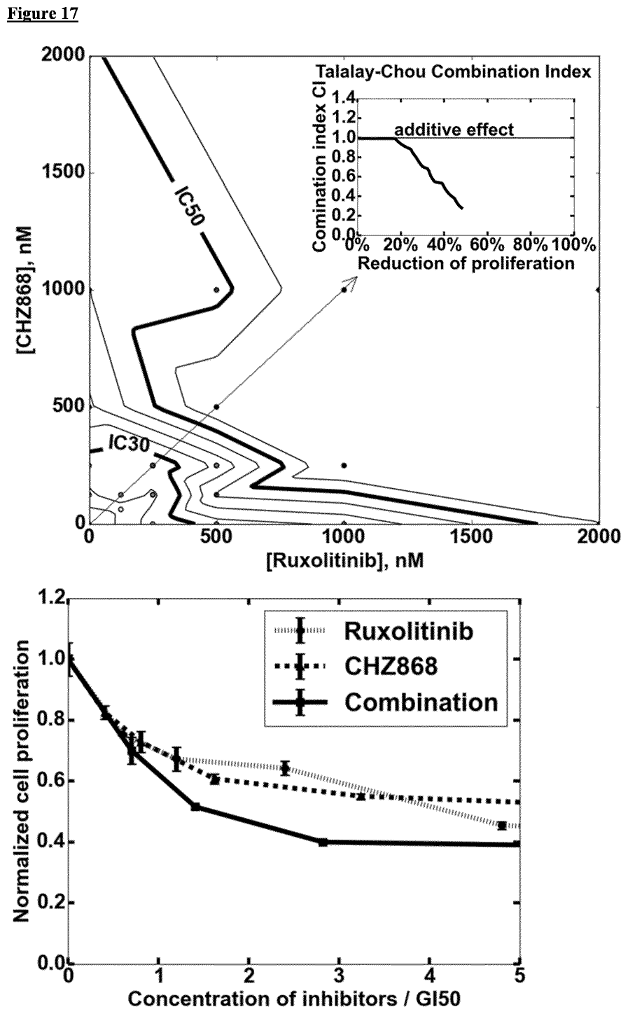

FIG. 17. Combination of Ruxolitinib and CHZ868 Inhibitors Synergistically Inhibit Proliferation of T-ALL leukaemia cells (DND41). (A, B) Proliferation of DND41 (T-ALL leukaemia) cells in response to Ruxolitinib (Type I), CHZ868 (Type II) and combination treatment (4 days). Shown is a representative of 3 biological replicates. Error bars indicate standard error of mean. Insert represents assessment of synergy strength assessed by the means of Talalay-Chou combination index for a specific directionratio of the combined drugs shown by arrow. The combination index shows marked synergy for intermediate levels of drugs.

### EXAMPLE 1

The following experiments were combined with a computational approach so as to analyze combinations of structurally different RAF inhibitors, which can efficiently suppress MEK/ERK signaling.

### RAF Inhibitors

Vemurafenib (PLX4032) was obtained from Selleckchem (Cat No. S1267). Sorafenib tosylate and SB-590885 were purchased from Axon Medchem (Axon 1397) and R&D Systems (2650/10), respectively. All inhibitors were dissolved in DMSO to yield 10 mM stocks and stored at −20° C.

Cell Culture Cell lines were either purchased from ATCC (SKMEL2, A375) or DSMZ (MEL-JSUO). M249 cells and the isogenic Vemurafenib-resistant cell line M249R were provided by Antoni Ribas (Nazarian et al., 2010). All cells were grown in RPMI (Gibco) supplemented with 2 mM L-glutamine and 10% (v/v) fetal bovine serum in a humidified atmosphere of 5% CO₂ at 37° C. Cells were seeded in 12-well plates (Greiner CELLSTAR dishes) at the density of 10⁵ cells per well. After reaching sufficient confluency, cells were treated with different concentrations of inhibitors and DMSO as control. To prepare the protein lysates, plates were transferred on ice, washed with ice-cold PBS and harvested by scraping in specific ELISA buffers as indicated below.

### MSD Multi-Spot Assay ELISA System

ERK and MEK activation was assessed by ELISA using the MESOSCALE MSD Phospho/Total ERK1/2 assay whole cell lysate Kit [phospho(Thr202/Tyr204; Thr185/Tyr187)/Total ERK1/2 Assay Whole Cell Lysate Kit, K15107D] or MEK kit [Phospho(Ser217/221)/Total MEK1/2 Assay Whole Cell Lysate Kit, K1512913] according to the manufacturer's instructions. Briefly, following the addition of complete MSD lysis buffer and scraping the cells from the surface of the dish, the cellular debris was removed from the lysate by centrifugation at 10000×g at 4° C. for 10 min. Protein concentration was determined using the BCA test according to the manufacturer's instructions (Pierce™ BCA Protein Assay Kit). Lysates were adjusted to 0.1 μg/μL protein concentrations for ERK kit and 0.8 μg/mL for MEK kit and relative MEK and ERK activation assessed according to the manufacturer's instructions using the MSD Sector Imager 2400 (model 1250).

xMAP Assays

Following the addition of complete Luminex lysis buffer and scraping the cells from the surface of the dish, the cellular debris was removed from the lysate by centrifugation at 10000×g at 4° C. for 10 min. The pellet was discarded and the protein concentration of lysates was adjusted to 0.3 μg/μL using the BCA assay kit. xMAP assays were performed on a Luminex-3D platform (Luminex, Austin, Tex.) using commercially available phosphoprotein antibody-coupled beads (ProtATonce, Athens, Greece). A custom multiplex phosphoprotein assay was used to determine the levels of test phosphoproteins in cell lysates: dual specificity mitogen-activated protein kinase kinase-1 (MEK1) with phosphorylation site S217/S221, and extracellular signal-regulated kinase-1 (ERK1) with phosphorylation site T202/Y204. Additionally, for loading control the levels of glyceraldehyde 3-phosphate dehydrogenase (GAPDH) protein were analyzed in a separate setting. Custom antibody-coupled beads were technically validated as described before (Poussin et al., 2014).

### Western Blot

Cells were transferred on ice, scraped using lysis buffer (10 mM Tris pH 7.5, 150 mM NaCl, and 0.5% (v/v) NP-40) complemented with protease and phosphatase inhibitors, and the cellular debris removed from the lysate by centrifugation at 10,000×g at 4° C. for 10 min. Protein concentration was determined using the BCA test according to the manufacturer's instructions (Pierce™ BCA Protein Assay Kit). Lysates were then resolved by SDS PAGE (12%) and transferred on a polyvinylidene difluoride membrane (Millipore). Protein visualization was performed in combination with horseradish peroxidase-conjugated secondary antibodies (Cell Signalling Technologies) and the enhanced chemiluminescence system (GE Healthcare) using the Advanced Molecular Vision Chemi Image Unit associated with ChemoStar Imager (INTAS Science Imaging Instruments GmbH) for the following antibodies: Polyclonal rabbit anti-human mitogen-activated protein (MAP) kinase [extra-cellular signal-regulated kinase (ERK) 1 and ERK2] antibody (Sigma), monoclonal mouse anti-human MAP kinase activated (diphosphorylated ERK1 and ERK2) antibody (Sigma).

### Cell Proliferation Assay

Cell proliferation was analyzed by CellTiter 96 Aqueous One Solution Cell Proliferation Assay (MTS; Promega) according to manufacturer's instructions. For this, 5,000 cells were plated per well of 96-well tissue culture plates (in 200 μL of medium). Proliferation and viability of inhibitor- and control-treated cells was assayed after 96 hrs. The results represent the mean±SD of triplicate samples, expressed as a percentage of control.

### Colony Formation Assay

For colony formation assay, 1,000 cells per well were seeded into 6-well plates and on the next day drug treatments were performed. Two weeks after the treatment, cells were fixed and stained using the Fixing/Staining solution (Crystal Violet (0.05% w/v), Formaldehyde (1%), PBS (1×), Methanol (1%)). Washed and air dried dishes were scanned and analysed by eye and using Clono-Counter software package (Niyazi et al., 2007).

### Molecular Dynamics Simulations

The ATP-pBRAF homodimer had been previously modeled using atomistic molecular dynamics (MD) simulations (Jambrina et al., 2016). Longer simulations were run and analyzed the dynamic adjustment of the αC-helix position by defining the w angle via the C and N terminal residues of the αC-helix (Ca atoms of Q493 and T508), the anchoring αF-helix (Ca atom of A641).

The initial conformation for the molecular dynamics (MD) simulation was based on the PDB structures 4E26 (Qin et al., 2012) corresponding to the active forms of the BRAF kinase domain. The ATP and the two Mg²⁺ ions were docked in the active site based on the 4DFX structure (Bastidas et al., 2012). The initial coordinates of the missing residues (439-447, and 604-609) were modeled using the M4T server (Fernandez-Fuentes et al., 2007a; Fernandez-Fuentes et al., 2007b; Rykunov et al., 2009). The homodimer system included a short, 20 amino acid-long substrate-like peptide (SP20 (Bastidas et al., 2012)) bound in the active site of each monomer. The ATP-pBRAF homodimer has the following phosphorylated activating residues: S446 in the NtA motif, T599 and S602 in the activation loop, and 5579 in the catalytic domain. The initial model was essentially symmetrical, the all-atom RMSD between the protomers was 0.07 Å and the dimer interactions are remarkably similar (all-atom RMSD=0.7 Å obtained for the alignment of the dimer but with the positions of the protomers inter-switched).

The simulation was performed using explicit water with the CHARMM-27 force field (MacKerell et al., 1998; Mackerell et al., 2004) at constant temperature (298 K) and pressure (1 bar). Langevin dynamics was used with a Langevin damping coefficient of 1 ps⁻¹. For long range electrostatics treatment, the non-bonded switching distance was set to 10 Å and a cut off distance of 12 Å was used. The ShakeH algorithm (Ryckaert et al., 1977) was used with 2 fs time steps. The trajectory was saved every 0.2 ns.

### Mathematical Model

The RAS/RAF/MEK/ERK pathway mathematical model was formulated using a rule-based approach (Chylek et al., 2014), in which protein-protein interactions are represented by rules. Each rule is associated with a rate law and defines a class of reactions related by a common transformation. The model was specified using BNGL, a formal language for writing rule-based models (Faeder et al., 2009). The model specification file, supplied in electronic format in the supplemental online material, was processed by the BioNetGen software package (Blinov et al., 2004; Harris et al., 2016) to derive the reaction network and the corresponding system of coupled ordinary differential equations (ODEs) implied by the rules. The ODEs were numerically integrated using BioNetGen's default algorithmic parameter settings and interface to CVODE in the SUNDIALS software package (Hindmarsh et al., 2005). Sensitivity coefficients of the model-predicted drug interaction metrics (CI and AUC) were calculated as the fractional change in the AUC or CI divided by the fractional parameter change (variation of the parameter change between 1 and 5% practically did not affect the sensitivity values).

### Exploiting RAF Dimer Asymmetry as Drug Target

The structural studies of the BRAF and CRAF kinase domains showed that dimers are asymmetric, and that RAF inhibitors often only bind one protomer. This asymmetry allows allosteric activation of a RAF protomer by a drug-bound protomer and is a critical feature of the paradoxical ERK pathway activation induced by many RAF inhibitors (Hu et al., 2013; Jambrina et al., 2014; Jambrina et al., 2016; Kholodenko, 2015; Yao et al., 2015). This asymmetry is hallmarked by different (IN-OUT) orientations of the αC-helix together with distinct IN and OUT conformations of the DFG motif (FIG. 1). These conformations occur naturally (FIG. 1B), but can be stabilized by RAF inhibitors, as suggested by crystallographic structures of BRAF with different inhibitors (FIG. 1B) (Rukhlenko et al., 2018).

Table 1 related to FIGS. 1 and 2. Summary of the structural properties of RAF inhibitors

| Type of | Orientation | Orientation of | | Examples of |
|---|---|---|---|---|
| inhibitor | of αC-helix | DFG-motif | Abbreviation | inhibitors |
| I | IN | IN | CI/DI | SB590885, |
| | | | | GDC-0879 |
| I½ | OUT | IN | CO/DI | vemurafenib, |
| | | | | dabrafenib, LGX818 |
| II | IN | OUT | CI/DO | sorafenib, AZ-628, |
| | | | | TAK-632, |
| | | | | LY3009120 |

These structural changes combined with the evidence from thermodynamic studies that dimerization can substantially change the affinity of protomers for a drug (Kholodenko, 2015), prompted the inventors to hypothesize that combining RAF inhibitors that preferentially bind to alternative αC-helix and DFG motif conformations should be able to block RAF dimer activity. As RAF dimerization involves not only conformational changes but also is governed by dynamic PTMs, which are difficult to track by structural and biochemical studies, the inventors developed an integrated computational model that allowed us to analyze both the phosphorylation and conformational dynamics in mechanistic detail.

### Structural, Thermodynamic and Kinetic Mechanisms Integrated in a Model

Protein functions are regulated by (de)phosphorylation of specific residues on multiple interacting, regulatory and catalytic domains (Pawson and Nash, 2003; Romano et al., 2014; Rubinstein et al., 2016). The ensuing protein states determine the affinities and rates of numerous interactions, including homo- and hetero dimerization, other protein associations, binding of inhibitors, and catalysis. To precisely account for the complexity of these interactions that occur sequentially or in parallel, the inventors implement a rule-based, domain-oriented approach, which explicitly monitors the conformational and phosphorylation states of pathway kinases, including inhibiting and activating phosphosites (Borisov et al., 2008; Chylek et al., 2014; Varga et al., 2017). The model describes conformational states of RAF monomers and dimers in terms of IN and OUT positions of the DFG motif and the αC-helix. These positions depend on RAF binding to RAS-GTP, the phosphorylation states of key residues (see below), the dimerization status (e.g., allosteric transactivation of a free RAF protomer by inhibitor-bound protomer (Hu et al., 2013)), and binding of RAF inhibitors that can stabilize the αC-helix and the DFG motif in the IN or OUT position, depending on the inhibitor structure. Each rule in inventors' RAS/ERK pathway model determines a set of chemical reactions, whose rates depend on the conformational, phosphorylation and spatial localization states of RAS, BRAF, CRAF, MEK and ERK. The main features of the complex RAF regulation, conformational transitions and allosteric interactions with RAF inhibitors that are integrated in the model are detailed below. A detailed list of assumptions, a description of processes and parameters, and a program file that can be processed by the open source software package BioNetGen (Chylek et al., 2014).

### RAF Activation Cycle

The model recapitulates how the activities of WT RAF kinases are controlled by (i) inhibitory phosphorylation on S259 for CRAF and 5365 for BRAF, (ii) activating phosphorylation on S338 for CRAF, (iii) homo- and heterodimerization, and (iv) inhibitory feedback phosphorylation by ERK on several sites (including S642 on CRAF and T753 on BRAF), as illustrated in FIG. 2 (Baljuls et al., 2013; Dhillon et al., 2002; Ritt et al., 2010). RAS-GTP is considered an input to the ERK cascade. In the absence of RAS-GTP, both CRAF and BRAF reside in the cytoplasm in inactive states characterized by pS259 (p denotes phosphorylation) and S338 for CRAF and pS365 for BRAF. Active RAS recruits CRAF and BRAF to the plasma membrane. This is followed by RAF conformational changes, the dissociation of 14-3-3 proteins, dephosphorylation of inhibitory pS259 or pS365, and phosphorylation of 5338, resulting in catalytic activity of RAF monomers (Chiloeches et al., 2001; Dhillon et al., 2002). Strikingly, catalytic activities increase more than 10-fold following RAF heterodimerization (Freeman et al., 2013; Rushworth et al., 2006). The details of the model are presented in (Rukhlenko et al., 2018).

### Influence of ERK Feedback on RAF Activity

In the model, ERK phosphorylation affects RAF activities through three different mechanisms. First, it lowers the binding affinities of both CRAF and BRAF for RAS-GTP (Dougherty et al., 2005; Ritt et al., 2010). Second, it dramatically decreases the activity of CRAF monomers (Dougherty et al., 2005). Third, ERK phosphorylation lowers the affinities of monomers to dimerize. This leads to dissociation of RAF dimers, resulting in a precipitous drop in the total kinase activity (Ritt et al., 2010; Rushworth et al., 2006). Owing to these mechanisms, the activity of RAF kinases is tightly controlled by ERK-mediated feedback in the absence of oncogenic RAS and BRAF mutations (Kholodenko and Birtwistle, 2009; Sturm et al., 2010).

### Oncogenic BRAF Mutant

Both WT RAF and mutant BRAFV600E (heterozygous and homozygous) cells were modeled. In the model BRAFV600E monomers are constitutively active, irrespective of the phosphorylation state of inhibitory S365, as suggested by structural and biochemical studies (Hu et al., 2013). Similar to wild-type, BRAFV600E is recruited to the plasma membrane by active RAS. The dimerization potential of mutant BRAF is higher than that of wild-type protein, and the stability of BRAFV600E dimers is less affected by ERK feedback phosphorylation than in the case of WT BRAF (Lavoie and Therrien, 2015).

Allosteric Interactions of RAF Monomers and Dimers with Inhibitors

Structurally diverse RAF inhibitors preferentially bind to different specific conformations of RAF molecules. Owing to thermal motions, these conformations can spontaneously transition between IN and OUT positions of the DFG motif and αC-helix (Lavoie and Therrien, 2015; Shao et al., 2017). Therefore, the apparent dissociation constants (K_{d}) of inhibitor binding to RAF monomers and dimers will depend on the equilibrium constants of these transitions, which in turn critically depend on the RAF binding, phosphorylation and dimerization states captured in the model. Distinct inhibitor types differentially stabilize IN or OUT positions of the αC-helix and the DFG motif and allosterically change these equilibrium constants and K_{d}'s (Kholodenko, 2015). Thus, a unique feature of the model developed is its inclusion of conformational transitions of the αC-helix and the DFG motif in kinase monomers and dimers, which are driven by the kinetics of RAF activation cycles, interactions with inhibitors and thermal intramolecular motions (illustrated in FIG. 2).

Summarizing, the model describes how the dynamic assortment of different RAF states determines the K_{d} of inhibitor binding within a cell. These K_{d} values critically depend on BRAF and RAS oncogenic mutations, thermal RAF motions, and the rate constants of inhibitor binding to different RAF conformations. Importantly, structurally diverse RAF inhibitors will have different K_{d}'s for different RAF molecular states, which is a prerequisite for inhibitor synergy or antagonism in cellular dose-responses. Next, using the comprehensive, structure-based model, combinations of structurally different RAF inhibitors can effectively suppress ERK signaling in cancer cells with distinct genetic and protein expression background will be assessed and then the model predictions tested in experiments.

BRAFV600E Mutant-Driven Cells with WT RAS

Signaling by BRAFV600E monomers is successfully blocked by RAF inhibitors that are used in the clinic, such as vemurafenib and dabrafenib. However, these drugs cannot effectively suppress signaling by RAF dimers, leading to paradoxical ERK activation and the emergence of resistance, when RAF dimerization is increased through different adaptive mechanisms. Importantly, model simulations suggest that in both homo- and heterozygous cells harboring mutant BRAF and WT RAS, BRAFV600E homo- and heterodimers considerably contribute to the total RAF activity (Rukhlenko et al., 2018). Therefore, synergy between RAF inhibitors will occur if they cooperate to efficiently inhibit RAF dimers (Kholodenko, 2015). The inventors' hypothesis is that two RAF inhibitors binding to alternative conformations of the αC-helix and/or DFG motif could block RAF dimer activity. Binding of a CO/DI inhibitor to an inhibitor-free RAF dimer stabilizes the dimer and the αC-helix of the respective protomer in an OUT position, whereas the αC-helix of the other protomer shifts to an IN position (because two αC-OUT protomer positions are generally incompatible with the dimer structure (Karoulia et al., 2016)). As drugs preferentially binding to an IN position of the αC-helix will select this protomer, a CO/DI and CI/DO inhibitor pair and a CI/DI and CO/DI pair may potentially synergize in the ERK pathway inhibition.

Using the model, the stationary dependencies of active MEK and ERK on the doses of RAF inhibitors was simulated, applied separately or in combination (these dependences are referred to as dose-responses). The levels of active MEK (ppMEK, FIG. 3A) and ERK (Rukhlenko et al., 2018) were normalized by their basal levels in growing cells, and drug exposure was simulated for several hours to reach the system steady state. To compare dose-response curves for different inhibitors, doses are commonly normalized by the IC50 values for each drug, which are the doses that inhibit the basal MEK or ERK activity by 50% (or by other ICZ values where 0≤Z≤100%) (Chou, 2006; Greco et al., 1995; Yeh et al., 2009). Accordingly, normalized dose-response curves for two different inhibitors always cross at the point where the normalized dose of each drug equals one (see blue and green dose-response curves in FIG. 3). Several quantitative metrics exist to estimate if two different inhibitors synergize, antagonize or act independently in suppressing pathway signaling. The Talalay-Chou combination index (CI) identifies drug synergy, additivity or antagonism, if the CI is smaller than 1, equal 1 or greater than 1, respectively (Chou, 2006). An advantage of using CI is the smaller amount of required data points, as compared with other, more comprehensive drug interaction metrics (see below for more details).

The simulations suggest that in BRAFV600E/WT RAS cells, two structurally distinct RAF inhibitors can synergize, if they preferably bind to protomers with different orientations of the αC-helix in a dimer (see the inserts in FIG. 3 panels showing that the CI is smaller than 1 over a range of doses). A combination of CO/DI and CI/DO inhibitors is most effective, suppressing ppERK with almost no paradoxical activation, whereas a combination of CO/DI and CI/DI inhibitors that can also be synergistic shows substantial paradoxical activation mainly induced by a CI/DI inhibitor (Rukhlenko et al., 2018). Also, in BRAFV600E/WT RAS cells, CI/DI and CI/DO inhibitors will not be synergistic, because BRAFV600E homodimers and BRAFV600E-BRAF dimers will be ineffectively inhibited by this drug pair (Rukhlenko et al., 2018). A comparison of the calculated dose-responses (FIG. 3A) with experimentally measured response curves in A375 (BRAF^{V600E/V600E}, WT RAS) cells (FIG. 3B) demonstrates that the model accurately predicts synergistic inhibition of the ERK pathway by sorafenib (CI/DO).

The synergy between CI/DO and CO/DI inhibitors increases, if a CO/DI inhibitor has a low dissociation rate constant (k_{off}), as, e.g., LGX818 with 1/k_{off}≥2 hrs (Yao et al., 2015). Strikingly, this low k_{off} does not change the efficiency of this inhibitor applied separately, but it markedly enhances the synergistic effect of the drug combination (Rukhlenko et al., 2018). Intriguingly, if a CI/DO drug has a low k_{off}, this almost does not affect the efficacy of this drug applied separately or in combination with a CO/DI drug (Rukhlenko et al., 2018).

Inhibition of Mutant RAS-Driven Cells with WT BRAF by Combinations of RAF Inhibitors

Specific RAF inhibitors used in the clinic are ineffective against tumors harboring oncogenic RAS mutations (Heidorn et al., 2010; Zhang et al., 2015). A combination of a RAF inhibitor (dabrafenib or vemurafenib) and a MEK inhibitor (trametinib) is the current standard of care for BRAFV600E-driven metastatic melanoma (Grob et al., 2015; Larkin et al., 2014). However, the model simulations suggest that this drug combination does not synergize to inhibit ERK signaling in oncogenic mutant RAS and WT BRAF cells (FIG. 4A). In fact, the model predicts that this combination increases the ppERK signal compared to MEK inhibitor alone (FIG. 4A). The inventors tested this prediction using the oncogenic RAS mutant-driven melanoma cell line MEL-JUSO (NRAS^{Q61L/WT} and HRAS^{G13D/G13D} (Forbes et al., 2015)). The experimental results corroborate model predictions, demonstrating that in MEL-JUSO cells the addition of dabrafenib to trametinib (at the doses that do not fully inhibit ERK activation) increases rather than decreases ERK signaling (FIG. 4B). Therefore next, the inventors explore whether RAF inhibitor combinations can effectively suppress ERK signaling in RAS mutant-driven cells.

### Combination of CI/DI and CI/DO RAF Inhibitors

Oncogenic RAS increases the abundance of BRAF-CRAF dimers. Because in a dimer, the BRAF protomer is dephosphorylated on S365, the equilibrium position of its DFG motif is shifted to the DFG-IN conformation. Consequently, CI/DI inhibitors preferentially bind to this BRAF protomer, stabilizing the DFG-IN conformation. Experimental data suggest that in growing cells the CRAF protomer is not phosphorylated on S338 in a considerable fraction of BRAF-CRAF dimers (Dhillon et al., 2002; Diaz et al., 1997), which is recapitulated in the simulations (Rukhlenko et al., 2018). Consequently, the DFG-motif of this CRAF protomer has a higher probability to be in an OUT position than in an IN position. As a result, this protomer will preferentially bind a CI/DO inhibitor, underpinning a potential synergy between CI/DI and CI/DO inhibitors. Importantly, this mechanism of synergy does not depend on which type of inhibitor binds first to a heterodimer; a CI/DI inhibitor would predominantly bind to a BRAF S365 protomer, whereas a CI/DO inhibitor would predominantly bind to a CRAF S259, S338 protomer. The model suggests that a substantial fraction of fully inhibited CRAF-BRAF heterodimers will contain a pair of CI/DO and CI/DI inhibitor molecules instead of two copies of either inhibitor (Rukhlenko et al., 2018). The simulated dose-responses show that either inhibitor induces a strong paradoxical ERK activation (Rukhlenko et al., 2018). In agreement with experimental studies (Karoulia et al., 2016), CI/DI inhibitors are predicted to show a higher paradoxical ERK activation than CI/DO inhibitors. Notably, the concentration ranges in which inhibitors lead to paradoxical activation become wider with increasing RAS-GTP levels (Rukhlenko et al., 2018).

The inhibitory effect of a two drug combination can be comprehensively assessed by calculating or measuring the ppERK response across a two-dimensional plane of drug doses, FIG. 5A (Keith et al., 2005; Yeh et al., 2009). Lines of constant ppERK inhibition are termed Loewe isoboles (Greco et al., 1995) (IC20, IC50 and IC80 isoboles are shown in FIG. 5A). For non-interacting drugs, these isoboles are straight lines. If two inhibitors synergize, Loewe isoboles are concave, since lesser doses result in the same inhibitory effect. Convex isoboles indicate antagonism between inhibitors, because their combinations require increased doses to achieve the same inhibition. Importantly, these distinctive features of Loewe isoboles do not depend on the normalization method (any ICZ value can be used), or even absolute, non-normalized inhibitor doses can be plotted. Different ratios result in different total doses for achieving the same ppERK inhibition. For each desired inhibition level (Z) there is a minimal total dose determined by an optimal ratio of drug doses, which together achieve the Z level of inhibition. Therefore, the commonly used 1:1 ratio of normalized inhibitor doses can be suboptimal for desired inhibition levels, which suggests that in preclinical studies, a two-dimensional plane of inhibitor doses might need to be analyzed.

Whereas in BRAFV600E/WT RAS cells, a combination of CI/DI and CI/DO inhibitors does not show synergy (Rukhlenko et al., 2018), this combination synergistically suppresses ERK activity in oncogenic RAS mutant, WT BRAF cells, FIG. 5A. The response plane in FIG. 5A shows that 2.2:1 ratio is optimal for achieving 80% inhibition of ppERK at the minimal total dose of both inhibitors. Following paradoxical ERK activity increase, the inhibitor combination becomes more effective than either inhibitor, starting at the doses around ½ of the IC50. For instance, when the sum of two inhibitor doses equals 1 (i.e., when each inhibitor is added at 0.5 of its IC50), the ppERK level drops more than 2-fold, compared to the level when each inhibitor is applied separately at the IC50 dose. At the same time, this inhibitor combination failed to considerably reduce paradoxical activation, suggesting that other inhibitor type combinations need to be also analyzed.

Synergy between CI/DI and CI/DO inhibitors strengthens when a CI/DO inhibitor has a low dissociation rate constant, k_{off}, such as TAK-632 and AZ-628 with l/k_{off}≥2 hrs (Hatzivassiliou et al., 2010; Okaniwa et al., 2013). After this CI/DO inhibitor binds to inactive RAF monomers, it facilitates RAF dimerization and remains bound, because of its low k_{off}. This leads to the accumulation of heterodimers where one RAF protomer is bound to a CI/DO inhibitor, whereas the other protomer is inhibitor-free (Kholodenko, 2015). An inhibitor-bound and kinase-inactive RAF protomer in a dimer allosterically transactivates the free RAF protomer, which then assumes an active DFG-IN conformation and has higher affinity for a CI/DI inhibitor than for the second CI/DO inhibitor molecule. Inventors' modeling results demonstrate that lowering k_{off} of a CI/DO inhibitor (while keeping the K_{d} value the same) markedly enhances synergy between CI/DI and CI/DO inhibitors but does not considerably change the efficiency of this inhibitor as a single agent (Rukhlenko et al., 2018).

### Testing Modeling Predictions for Oncogenic RAS Mutant Cells

To test model predictions, experiments in the MEL-JUSO (NRAS^{Q61L/WT} and HRAS^{G13D/G13D}) melanoma cell line (FIG. 5B) were conducted. In these cells, the dose-responses of active MEK and ERK to increasing doses of SB-590885 (CI/DI RAF inhibitor (Heidorn et al., 2010)) and sorafenib (CI/DO RAF inhibitor (Heidorn et al., 2010; Holderfield et al., 2014)) added separately or in combination were measured. Experimental data allowed reconstruction of a substantial part of the dose-response plane across multiple inhibitor combinations (FIG. 5B). Responses to each inhibitor show marked paradoxical ERK activation, extending into the micromolar range for either inhibitor, while in in vitro kinase assays both inhibitors inhibit all RAF isoforms in the low nM range (King et al., 2006; Wan et al., 2004; Wilhelm et al., 2004). Because in mutant NRAS and HRAS MEL-JUSO cells, SB-590885 did not suppress ERK activity (in the dose range used), it was not possible to normalize inhibitor doses by commonly used IC50 levels, and plot responses versus absolute inhibitor doses. Therefore, for each ppERK response to a combination of SB-590885 and sorafenib shown on the dose-response plane in FIG. 5B, the total inhibitor dose is the sum of the absolute SB-590885 and sorafenib concentrations (that can be found by projections of the corresponding ERK response point onto axes). The concave shapes of the Loewe isoboles (lines of constant ppERK inhibition) in FIG. 5B confirm inventors' model predictions, demonstrating marked synergy for the combination of SB-590885 with sorafenib. The optimal ratio of sorafenib to SB-590885 doses to achieve 75% ppERK inhibition was about 1.5:1.

When the number of data points across the two-dimensional plane of inhibitor doses is insufficient to reconstruct the Loewe isoboles, the combination index CI is commonly used to identify synergy or antagonism (Chou, 2006). For any particular drug combination ratio, the CI detects if at this ratio the Loewe isoboles will be concave (under a straight line of non-interacting drugs), in which case CI<1, or convex (above this line), in which case CI>1. Importantly, the classic metrics for assessing drug interactions, such as the Chou combination index or Loewe isoboles cannot apply to the range of doses, at which individual inhibitors and their combinations paradoxically activate a pathway. An objective measure of suppressing pathway signaling is the area under the dose response curves for each inhibitor taken separately and in combination (Kholodenko, 2015). The data presented in (Rukhlenko et al., 2018) demonstrate that this area and therefore, resistance to inhibition, substantially diminishes for a combination of CI/DI and CI/DO inhibitors.

### Combination of CI/DO and CO/DI RAF Inhibitors

Next, in oncogenic RAS mutant cells inventors analyzed combinations of RAF inhibitors that preferably bind distinct orientations of both DFG motif and αC-helix. These drugs, and also inhibitors that preferentially bind only distinct αC-helix orientations, can potentially synergize in both BRAFV600E/WT RAS cells and in RAS mutant cells. Yet, model simulations show that the best combination for RAS mutant cells is a pair of CI/DO and CO/DI inhibitors (FIG. 6A), whereas a pair of CI/DI and CO/DI inhibitors induces marked MEK/ERK paradoxical activation (Rukhlenko et al., 2018). Experiments in MEL-JUSO (NRAS^{Q61L/WT}, HRAS^{G13D/G13D}) and SKMEL2 (NRAS^{Q61R/WT}) cells, bearing an activating RAS mutations and WT BRAF, have collaborated modeling predictions that combinations of CO/DI and CI/DO inhibitors are more efficient than either of inhibitors alone, sorafenib (CI/DO) and their combination, and FIG. 6B for ppERK dose-responses in MEL-JUSO cells treated with vemurafenib (CO/DI), sorafenib (CI/DO) and their combination).

Interestingly, vemurafenib applied in doses up to 50 μM could only activate ppERK in MEL-JUSO cells (FIG. 6B), as reported for other RAS-mutant cancer cells (Adelmann et al., 2016; Karoulia et al., 2016). Sorafenib applied separately could only slightly inhibit ppERK at high doses (12 μM), following paradoxical activation. Remarkably, a combination of vemurafenib and sorafenib could effectively inhibit the ERK pathway (following paradoxical ERK activation) at the total doses over 8 μM (5 μM vemurafenib and 3 μM sorafenib). In line with inventors' model predictions, even RAF inhibitors, which on their own only activated ERK signaling, could inhibit the pathway when given in a proper combination.

Inhibition of Oncogenic RAS Signaling Correlates with Reduced Cell Proliferation and Colony Formation

A combination of CI/DO and CO/DI RAF inhibitors blocked oncogenic RAS signaling in MEL-JUSO cells. Therefore, next inventors explored how these combinations affect cell proliferation and colony formation potential, which tests for the ability of a single cell to survive and grow into a colony. Both vemurafenib and sorafenib applied individually inhibited MEL-JUSO cell proliferation with the GI50 (a dose inhibiting cell proliferation by 50%) of 32 μM and 8 μM, respectively (FIG. 6C). At 1:1 ratio, a combination of these drugs synergistically inhibited proliferation. When both drugs were combined at 50% of the corresponding GI50 dose, the combination inhibited the cell growth 2-fold more efficiently than each drug at its GI50 dose. Moreover, both drug synergy metrics, the CI and AUC, demonstrated a pronounced synergy between vemurafenib and sorafenib in inhibiting MEL-JUSO cell proliferation (insert to FIG. 6C demonstrates that the CI for inhibition of proliferation was smaller than 0.6 over a range of doses). Likewise, a combination of vemurafenib and sorafenib synergistically inhibited colony formation in MEL-JUSO cells (FIG. 6D). Inventors' data demonstrate that oncogenic RAS signaling, proliferation and the ability to form colonies were synergistically inhibited by a combination of CI/DO and CO/DI RAF inhibitors in MEL-JUSO cells (NRAS^{Q61L/WT}, HRAS^{G13D/G13D})

### Combinations of RAF Inhibitors Suppress ERK Signaling in Cells Bearing Both Oncogenic RAS and BRAFV600E Mutations

One of the common mechanisms of resistance to RAF inhibitors in BRAFV600E melanomas is the appearance of a secondary NRAS mutation in the ERK pathway (Johnson et al., 2015; Lito et al., 2013; Nazarian et al., 2010). Some melanoma patients develop secondary malignancies from cells harboring pre-existing RAS mutations, whereas for others, RAS mutations frequently occur during treatments with BRAF inhibitors (Nazarian et al., 2010). Instructively, the model predicts that inhibitor combinations that synergistically suppress ERK signaling in RAS mutant cells also synergistically inhibit ppERK in co-mutated RAS and BRAF600E cells (FIG. 7A). This model prediction is explained by the enhanced RAF dimerization and the fact that emerging dimers can be effectively inhibited only by a combination of RAF inhibitors. A combination of a CI/DO inhibitor with a low k_{off} CO/DI inhibitor is predicted to be particularly effective in suppressing ERK activity in these cells (Rukhlenko et al., 2018).

To test these predictions, parental (BRAF^{V600E/WT}/WT RAS) and vemurafenib resistant M249 (BRAF^{V600E/WT}/NRAS^{Q61K/WT}) cells (Nazarian et al., 2010) were treated with vemurafenib alone, sorafenib alone and the combination of these drugs. The data confirm that a combination of CI/DO and CO/DI inhibitors effectively suppresses ERK signaling in cells bearing both RAS and RAF oncogenic mutations (FIG. 7B). NRAS mutation results in about 3.5-fold increase in the basal ppERK level compared to parental cells (FIG. 7B). After treatment with 3 μM vemurafenib, the ppERK level in resistant M249 cells reaches the value equal to the basal level in the parental cells. Increasing the doses of vemurafenib from 3 to 10 μM does not substantially decrease the ppERK level in M249 cells. Although resistant to vemurafenib, ERK signaling is effectively inhibited in these cells by a 1:1 molar combination of vemurafenib and sorafenib starting from a total drug concentration of 3 μM. These results support model predictions.

### Robustness of Model Predictions

The predictive power of the structure-based, dynamic model of ERK signaling and inhibitor—kinase interactions was tested against experiments and corroborated by the resulting data. Yet, a question arises of how robust these model predictions are, when one went beyond the possibilities of direct experimental testing. To answer this question, inventors carried out the sensitivity analysis of model-predicted drug interaction metrics to the changes in model parameters were carried out. The area under dose-response curves (AUC, an objective measure of pathway inhibition for a range of drug doses) was explored, and the Talalay-Chou combination index (CI) are sensitive to parameter changes, by calculating the response coefficients, Rₚ^{AUC} and Rₚ^{CI}. These response coefficients (also known as the control or sensitivity coefficients, see, e.g., (Kholodenko et al., 1987; Kholodenko et al., 1997; Kholodenko and Westerhoff, 1995)) determine the fractional change in the AUC and CI brought about by a small fractional change in a model parameter p, which in the limit of infinitesimal changes reads, Rₚ^{X}=lim((ΔX/X)/(Δp/p))=d ln X/d ln p, X={AUC, CI}. Thus, Rₚ^{AUC} and Rₚ^{CI} are essentially equal the % changes in the AUC and CI caused by a 1% change in a parameter. If Rₚ^{AUC} and Rₚ^{CI} are substantially smaller than 1, the model predictions are robust to the changes in the corresponding parameter.

The robustness of model predictions for two types of drug resistant melanoma cells, harboring either oncogenic RAS mutations and WT BRAF (MEL-JUSO, SKMEL2) or bearing both oncogenic RAS and heterozygous BRAFV600E mutations (vemurafenib resistant M249 cells) was explored in (Rukhlenko et al., 2018).

### CONCLUSIONS

To describe the experimental data the authors designed a simplified kinetic model that correlates changes in phosphorylation of the EGFR with drug binding without elaborating the underlying molecular mechanisms. A next-generation pathway model was developed that allows mechanistic and predictive analysis by dynamically integrating thermodynamics and kinetics of drug interactions, structural elements, PTMs, mutational status and pathway regulation. This model unravels salient features of the systems-level dose-responses to different types of RAF inhibitors that show similar inhibition of isolated RAF kinases, but preferentially bind to alternative conformations of the DFG motif and αC-helix adopted by RAF kinases as a result of different oncogenic activation mechanisms. Previous attempts of predicting dose-responses failed (Costello et al., 2014; de Gramont et al., 2015; Prasad, 2016; Saez-Rodriguez et al., 2015), because both the employed network models and machine learning methods could not embrace highly dynamic nature of allosteric interactions of structurally different drugs with multiple kinase conformations governed by thermal motions and posttranslational modifications (Nussinov et al., 2013). The type of next-generation models presented here can be instrumental in the future analysis of mechanisms of drug actions and the design of efficacious combinations. For instance, this approach could be extended to optimize combinations of RAF and MEK inhibitors.

The model explores RAF inhibitor combinations, it is based on general principles applicable to any kinases that undergo dimerization during activation (Bessman et al.). The model makes a surprising prediction that two drugs targeting the same protein pocket can synergize, while normally they would compete, as known from enzyme kinetics. However, a reason for potential synergy is asymmetry of protomer conformations that is induced by PTMs and/or binding of the first inhibitor molecule to a dimer (Jambrina et al., 2016; Kholodenko, 2015). These unexpected results would not have been discovered without mathematical and structural modeling, accounting for the asymmetry of protomer conformations in a kinase dimer. The model precisely predicts for which mutational profiles and which drugs will preferentially bind different protomers in a kinase dimer and together completely inhibit these dimers. This suggests an alternative principle that two structurally different inhibitors, which target the same kinase, but in different conformations, can be synergistic.

Different mechanisms of intrinsic or acquired resistance in melanoma have a common feature of the increased abundance of RAF dimers. Moreover, recent clinical sequencing of 10,000 metastatic cancers (Zehir et al., 2017) not only revealed the relatively common co-occurrence of NRAS and BRAF mutations that increase RAF dimerization, but also BRAF in-frame deletions, which produce isoforms predicted to enable RAS-independent BRAF dimerization similar to the BRAF splice variants previously associated with acquired resistance to vemurafenib (Poulikakos et al., 2011). Whereas pharmacological research concentrated on creating RAF inhibitors that do not induce RAF dimers and thereby avoid paradoxical ERK activation (Zhang et al., 2015), inventors' model suggested exploiting structural and thermodynamic features of dimer-drug interactions to completely inhibit RAF dimers. Based on model predictions, inventors showed that both BRAFV600E monomers and RAF dimers are best inhibited together by specific combinations of RAF inhibitors, even when each inhibitor is ineffective on its own. Importantly, the total dose of two combined drugs is considerably smaller than the dose of each inhibitor, which could substantially reduce toxicity resulting from off-target effects.

Experiments corroborate model predictions. In cancer cells bearing BRAFV600E mutation and WT RAS (A375 cell line, FIG. 3), BRAFV600E and NRAS Q61K co-mutations (resistant M249 cell line, FIG. 7) or oncogenic RAS and WT BRAF (MEL-JUSO cells, FIG. 6), a combination of CI/DO and CO/DI inhibitors showed pronounced synergy, effectively inhibiting ERK activation. The results suggest that for mutant BRAFV600E-driven cells, adding a CI/DO inhibitor (e.g., sorafenib, AZ-628, TAK-632, LY3009120) to a standardly used CO/DI inhibitor (vemurafenib, dabrafenib or encorafenib) can be beneficial not only because of more effective inhibition of ERK signaling in WT RAS cancer cells, but also because of synergistic inhibition of signaling in pre-existing or emerging resistant cancer cell clones with both BRAFV600E and RAS mutations. For these mutational profiles, especially for cells with mutant BRAF600E and WT RAS, a combination of CI/DI and CI/DO inhibitors is predicted to show additive rather than synergistic effects (Rukhlenko et al., 2018). Almost counterintuitively, the model predicts, and experiments confirm that the same combination of CI/DI and CI/DO inhibitors is markedly synergistic in oncogenic RAS mutant cells with WT BRAF (MEL-JUSO cells, NRAS^{Q61L/WT} and HRAS^{G13D/G13D}), FIG. 5. The combinations of RAF inhibitors described above can also be effective in suppressing RAF/ERK signaling in cells with other mechanisms of resistance, such as CRAF/BRAF overexpression and BRAF splicing variants that enable RAS-independent BRAF dimerization (Rukhlenko et al., 2018). Summarizing, although in cells bearing oncogenic RAS mutations, individual RAF inhibitors are commonly ineffective, proper combinations of RAF inhibitors with particular modes of action efficiently inhibit ERK signaling. Biologically, this effective ERK inhibition is accompanied by a synergistic suppression of proliferation and colony formation in MEL-JUSO cells (FIG. 6).

Similarly, modeling can also address the open question whether RAF inhibitors increase the affinity of RAF kinases for RAS. RAF inhibitors increase the amount of RAS-RAF complexes (Hatzivassiliou et al., 2010; Karoulia et al., 2016), which was interpreted as the facilitation of RAF binding to RAS-GTP by these drugs. Although this explanation is plausible, structural evidence is lacking. Moreover, the model demonstrates that allosteric inhibitor effects resulting in enhancement of RAF dimerization can fully explain the increase in RAS-RAF complexes without an assumption that RAF inhibitors increase RAF affinities for RAS-GTP (Rukhlenko et al., 2018). Because each of the RAF protomers in a RAF dimer is bound to RAS in the narrow layer near the membrane, the apparent affinity of RAF for RAS increases due to spatial localization effects. Also, recent data on RAS dimerization (Nan et al., 2015) suggest that the increase in the apparent affinity of RAF for RAS can be explained by spatial localization.

Inventors' combined modeling and experimental data also suggest novel potential treatment options for RASopathies (extensively reviewed in (Aoki et al., 2016; Jindal et al., 2015; Rauen, 2013; Rauen et al., 2011; Tajan et al., 2018)). RASopathies are a group of developmental disorders caused by germline mutations in various genes encoding components of the human RAS/RAF/MEK/ERK-MAPK pathway. They represent the largest group of multiple congenital anomaly syndromes and are characterised by a broad spectrum of morphological and functional abnormalities. The group of disorders include Neurofibromatosis type 1, Noonan syndrome, Noonan syndrome with multiple lentigines, capillary malformation-arteriovenous malformation syndrome, Costello syndrome (CS), cardio-faciocutaneous syndrome (CFCS), and Legius syndrome (Rauen, 2013). With its important role in cellular signalling and control of proliferation, growth, differentiation, and senescence, the underlying germline mutations dysregulating the MAPK pathway exhibit a number of overlapping phenotypic features and effects on development.

With numerous established therapeutic targets in this signalling pathway (RAF, MEK) for other diseases including cancer, a number of anti-cancer therapies are currently being tested in preclinical and clinical studies for repurposing for the treatment of RASopathies. However, to inventors' best knowledge, no drugs targeting the RAS-RAF-MAPK pathway are currently approved for the treatment of RASopathies.

In anti-cancer therapies, near maximum tolerated doses are applied short-time with cytotoxic effects, which are often accompanied by substantial side effects. In stark contrast, the treatment of RASopathies caused by germline mutations would require long-lasting and chronic treatment regimens with drug dosages aimed at normalizing the MAPK activation (Tajan et al., 2018). This would require drug doses below the cytotoxic targeted cancer therapies thereby limiting adverse side effects and allowing for long-term treatment.

Inventors' observed modelling and experimental on drug synergism of RAF inhibitors in NRAS- and BRAF-mediated malignant melanoma are strongly in line with these current ideas and ongoing trials for targeted approaches and therapies against a number of RASopathies.

Inventors claim that combinatorial treatment with synergistic structurally diverse RAF inhibitors is advantageous for the treatment of RASopathies allowing for lower doses of RAF inhibitors therefore limiting toxicities. With RAF dimerization known to be crucial for the great majority of physiological RAF-MAPK signalling functions, synergistic RAF inhibitor combinations could be applied to all RASopathies with germline mutations in RAF kinases (RAF1 and BRAF) themselves or upstream of RAF, such as HRAS, NRAS, KRAS, SOS1, SHOC2, RASA and NF1 mutations. These include but are not limited to Neurofibromatosis type 1, Noonan syndrome, Noonan syndrome with multiple lentigines, capillary malformation-arteriovenous malformation syndrome, Costello syndrome, cardio-faciocutaneous syndrome, and Legius syndrome (Rauen, 2013).

In summary, the type of next generation dynamic model presented developed can address salient issues in drug targeting as well as help discover new aspects of drugs mode of action. These insights can be exploited to rationally design drug combinations that would be difficult to find through trial-and-error approach.

### EXAMPLE 2

Effective and Synergistic Inhibition of the ErbB Receptor Family by Two Inhibitors with Different Conformation Selectivity

The kinase activity of the ErbB family receptor tyrosine kinases (RTKs) is triggered by the ligand-induced homo- and hetero-dimerization, oligomerization and subsequent conformational changes, whereas autophosphorylation of the activation loop is not required (Endres et al., 2011; Jura et al., 2011). The ErbB receptors form both symmetric and asymmetric dimers. Symmetric, head-to-head dimers are kinase inactive and are formed in the absence of a ligand or when only a single ligand molecule is bound to a dimer. Asymmetric dimers are kinase active and only formed when both ErbB molecules are bound to a ligand. In an asymmetric dimer, the activity of one kinase domain (acceptor of activation) is stimulated by binding to the other kinase domain (donor of activation) resulting in the phosphorylation of both C-terminal tails of ErbB receptors (Arteaga and Engelman, 2014; Endres et al., 2011; Jura et al., 2011; Macdonald-Obermann et al., 2013). Oligomerization or clustering of dimers increase their kinase activity (Claus et al., 2018).

The inventors have developed a mathematical model, demonstrating main features of dimerization of ErbB family receptor tyrosine kinases and their allosteric interactions with ATP-competitive inhibitors. The model predictions are corroborated by inventors' experimental data (see below, FIGS. 12-15). A simplified kinetic scheme of the dimerization model is presented in FIG. 8A. Here, R is an ErbB family receptor, e.g., EGFR for illustrative purposes, E is a ligand (e.g., EGF), R-R and R_{D}E-R_{A}E are symmetric and asymmetric dimers, and R_{D} is activation donor and R_{A} is acceptor. Asymmetric R_{D}E-R_{A}E dimer is kinase active and autophosphorylates the receptor cytoplasmic tails on multiple tyrosine residues that bind numerous adaptor and signalling proteins propagating the signal into a cell. In contrast, the kinase activity of a symmetric dimer with no bound ligand or a single bound ligand molecule is negligibly low.

The fundamental thermodynamic laws imply that dissociation constants of all 6 reactions presented on FIG. 8A can be expressed through the following two dissociation constants: 1). Dissociation constant of ErbB dimerization in the absence of ligand (K_{dim}), 2). Dissociation constant of ligand binding to ErbB monomer (K_{E}), and two thermodynamic factors describing binding of the first (f_{E}) and second (g_{E}) molecule of a ligand to an ErbB dimer (Kholodenko, 2015).

Thermodynamic factor f_{E} describes the change in the affinity of a ligand for a free ErbB dimer in comparison to the affinity for an ErbB monomer (FIG. 8). Because ligands stabilize ErbB dimers, inducing dimerization (Arteaga and Engelman, 2014; Endres et al., 2011; Jura et al., 2011; Macdonald-Obermann et al., 2013), the factor f_{E} is lower than 1. Thermodynamic factor g_{E} describes the change in the affinity of a ligand for an ErbB dimer already harbouring one bound ligand molecule as compared to the ligand affinity for an ErbB monomer. The factor g_{E} is bigger than f_{E} and greater than 1, because of the reported strong negative cooperativity of ligand binding to ErbB receptors (Arteaga and Engelman, 2014; Endres et al., 2011; Jura et al., 2011; Klein et al., 2004).

Kinase domains of the ErbB receptors toggle between inactive and active conformations that differ by the positions of regulatory motifs, the highly conserved DFG (Asp-Phe-Gly) motif and the αC-helix. ATP-competitive ErbB inhibitors can be classified based on their preferential binding to the different (IN or OUT) positions of these motifs. Type I inhibitors bind an active kinase conformation in which the active site DFG motif is in the ‘IN’ position (DFG-IN) and the αC-helix is also ‘IN’ (αC-IN). Type I1/2 inhibitors bind an active-like (but inactive) conformation with DFG-IN, and αC-OUT. Type II inhibitors bind the inactive DFG-OUT conformation, with αC either ‘IN’ or in an intermediate position (for RAF) or ‘OUT’ (for other kinases) (Fabbro, 2015; Roskoski, 2016). In a symmetric dimer both kinase domains predominantly occupy inactive DFG-OUT, αC-OUT position. In an asymmetric dimer, a donor kinase predominantly occupies a DFG-OUT, αC-OUT position, whereas the acceptor kinase mainly occupies a DFG-IN, αC-IN position (Arteaga and Engelman, 2014; Endres et al., 2011; Jura et al., 2011).

Importantly, small-molecule ErbB inhibitors induce dimerization of ErbB receptors both in the presence and in the absence of ligands (Macdonald-Obermann et al., 2013).

Kinetic schemes of binding of small-molecule inhibitors and ErbB dimerization are presented on FIGS. 8B-D and 9, kinetic schemes of binding ligands and inhibitors to ErbB dimers are presented on FIGS. 8E and 9E-F.

Using the detailed balance principle (Kholodenko, 2015), the inventors have expressed the dissociation constants of all reactions depicted in FIGS. 8B-E in terms of the dimerization constant (K_{dim}), the dissociation constant (K_{E}) of ligand binding to a free ErbB monomer, the dissociation constant (K₁) of binding to a free or ligand-bound monomer for inhibitor (I) and the following 4 thermodynamic factors. Thermodynamic factor f₁ describes facilitation of ErbB dimerization by inhibitor. Thermodynamic factor g₁ describes asymmetry of the dimer, and factors g₂^{S} and g₂^{A} describe the change in the dimer affinity for the second molecule of inhibitor for symmetric and asymmetric dimers, respectively. Thermodynamic description of allosteric interactions of symmetric and asymmetric ErbB dimers with two inhibitors (Iₐ, I_{b}) require additional thermodynamic factors, presented in the scheme in FIG. 9. The inventors have developed a mathematical approach that allows us to calculate the dissociation constants of inhibitors binding to different kinase forms based on information about the equilibrium constants of intra-molecular motions for this kinase. Using this approach and estimating the equilibrium constants of DFG-motif and αC-helix transitions for ErbB monomers and dimers based on the literature data, the inventors have derived expressions for dissociation constants to all monomeric and dimeric forms of ErbB receptors.

Using the inventors approach of structure-based modelling, the values of all thermodynamic factors except dimerization facilitation factor f₁ (that is experimentally determined (Macdonald-Obermann et al., 2013)) were derived from the equilibrium constants of DFG-motif and αC-helix transitions. In short, based on available literature data equilibrium constants of DFG-motif and αC-helix transitions were estimated for every monomer and dimer state of ErbB. The apparent dissociation constants (K_{d}^{app}) for all forms of ErbB molecules were calculated from the following equation:

$${K}_{d}^{app} = \frac{γ bc + b + c + 1}{\frac{γ bc}{{a}_{11}} + \frac{b}{{a}_{12}} + \frac{c}{{a}_{21}} + \frac{1}{{a}_{22}}} \cdot {K}_{d}$$

Here b and c are equilibrium constants of DFG-motif and αC-helix transitions, respectively, and γ is the coefficient of cooperativity between these transitions; K_{d} is the microscopic dissociation constant for inhibitor binding to the preferred kinase conformation; coefficients a₁₁, a₁₂, a₂₁ and a₂₂ describe inhibitor selectivity for IN and OUT positions of the DFG-motif and αC-helix where the first index corresponds to a position of DFG-motif (1-IN, 2-OUT), and the second index corresponds to ae position of αC-helix.

The computational model demonstrates profound synergy effects between type I and type I1/2 ErbB family inhibitors, as well as between type I and type II inhibitors for the suppression of the ErbB activity (see FIGS. 10A and 11A). The Talalay-Chou combination index captures this marked synergism, especially between type I and type I1/2 inhibitors. An underlying mechanistic reason of the predicted synergy effects is the asymmetry of an active ErbB dimer. Thus, asymmetric ErbB dimers are better inhibited by a combination of ErbB inhibitors than by any of such inhibitors applied alone. Importantly, that the total drug dose for an inhibitor combination is the same as the dose for each inhibitor applied separately (doses are normalized by IC50).

Importantly, the abundance of ErbB receptor molecules at the plasma membrane is constant during only relatively short periods of time. Treatment with ErbB family inhibitors is known to increase the abundance of ErbB receptors and the concentrations of their homo- and hetero-dimers at the plasma membrane (Arteaga and Engelman, 2014; Garrett et al., 2011; Scaltriti et al., 2009). This increase in the concentration of ErbB receptors results in partial reactivation of ErbB signalling. An ErbB inhibitor of any type is unable, on its own, to effectively bind to both protomers in an asymmetric ErbB dimer, because only one conformation (of either donor or acceptor) will be preferable for that particular inhibitor type. Importantly, when type I and type I1/2 or type I and type II inhibitors are used in combination, each of these structurally different inhibitors will bind the corresponding protomer conformation in an asymmetric dimer. Therefore, the increase in the ErbB abundance due to cellular resistance mechanisms will lead to significantly lower increase in ErbB phosphorylation when a combination of two inhibitors is applied compared to any single inhibitor (FIGS. 10B and 11B). Thus, the inventors' model predicts that treatment with combinations of structurally different ErbB inhibitors will inhibit signalling downstream of ErbB receptors much more robustly better than any of inhibitors on its own.

The experiments conducted by the inventors in HER2-positive breast cancer cell lines SKBR3, BT474 and AU565 corroborated modelling predictions (FIGS. 12-14). Similarly as demonstrated in recent work (Claus et al., 2018), the inventors observed that stimulation with Heregulin (HRG) renders HER2-positive breast cancer cells resistant to Lapatinib treatment. Thus, in order to model conditions of resistance to ErbB inhibitors, all proliferation assays were performed in the presence of HRG. The inventors have assessed cell proliferation of these three cell lines treated with Type I1/2 inhibitor Lapatinib, Type I inhibitors Gefitinib and Elrlotinib, and to a combination of Type I1/2 and Type I inhibitors.

Covalent inhibitors (Fabbro, 2015; Roskoski, 2016) can be modelled as type I, type I1/2 or type II inhibitors with very small dissociation kinetic constant k_{off}. Since the model-predicted synergy effects are mainly based on dimer asymmetry, the model also predicts and experiments corroborate (FIG. 15) synergy effects between covalent inhibitors, if they bind to different conformations of ErbB receptor tyrosine kinases. Thus, the inventors' model predicts synergy between covalent ErbB inhibitors that preferentially bind to DFG-IN, αC-IN conformation and either DFG-IN, αC-OUT or DFG-OUT, αC-IN/OUT conformations. Inventors' experiments demonstrate synergistic effects between Type I1/2 inhibitor Lapatinib and covalent inhibitor Afatinib, which binds to the kinase active (DFG-IN, αC-IN) conformation.

### EXAMPLE 3

Effective and Synergistic Inhibition of JAK1-JAK2 Heterodimers by Two Inhibitors with Different Conformation Selectivity

The identification of somatic activating mutations in JAK family kinases (Baxter et al., 2005; James et al., 2005; Kralovics et al., 2005; Zhao et al., 2005) and in the thrombopoietin receptor (MPL) (Pikman et al., 2006) in most patients with myeloproliferative neoplasm (MPN) led to the development and clinical use of JAK2 kinase inhibitors (Pardanani et al., 2011; Verstovsek et al., 2010). JAK2 inhibitor therapy improves MPN-associated splenomegaly and systemic symptoms but does not significantly decrease or eliminate the MPN growth in most patients. Resistance to JAK2 inhibitor therapy was found to be associated with heterodimerization between activated JAK1, JAK2 and TYK2 (Koppikar et al., 2012). Activation of JAK2 in trans by other JAK kinases in a heterodimer leads to reactivation of JAK-STAT signalling. RNA interference and pharmacological studies show that JAK2-inhibitor-resistant cells remain dependent on JAK2 protein expression (Koppikar et al., 2012). Mutations in JAK-associated receptors (e.g. IL7R) and JAK kinases which lead to hyper-activation of JAK-STAT pathway are known to be drivers of a group of blood cancers known as leukaemias. Consequently, therapies that would result in robust inhibition of JAK-STAT pathway could be beneficial to patients with JAK-dependent malignancies.

Cell line experiments show that resistance to JAK inhibitors develops in JAK inhibitor sensitive cells during 4-6 weeks of treatment with JAK inhibitors (Koppikar et al., 2012). Parental cells are sensitive to JAK inhibitors, both JAK-STAT pathway and their proliferation are suppressed by JAK inhibitors. In such parental cells, no significant JAK2-JAK1 hetero-dimerization was observed. In resistant cells, both JAK-STAT pathway and their proliferation are inhibited only by ˜1000-fold higher doses of JAK inhibitors, and this resistance is mediated by JAK2-JAK1 or JAK2-TYK1 hetero-dimerization (Koppikar et al., 2012). Inventors' model analyses symmetric and asymmetric JAK homo- and hetero-dimerization.

Thermodynamic principles explain resistance to JAK inhibitor therapy. These principles also allow us to express multiple reactions of JAK dimerization and inhibitor binding through the dissociation constant of JAK homo- and hetero-dimerization without inhibitors (K_{dimS} and K_{dimA}), the dissociation constants of inhibitor binding to JAK2 and JAK1 monomers (K_{I} and K_{I1}), and thermodynamic parameters. Activation of JAK kinases occurs at the plasma membrane where they bind to different hormone and cytokine receptors. These receptors induce homo- or hetero-dimerization of JAK family kinases. In the absence of ligands, a JAK dimer resides in a kinase-inactive state being bound to a receptor dimer. Binding of a ligand to a receptor dimer switches a JAK dimer to a kinase active state, which leads to phosphorylation of the activation loop of JAK kinases and JAK activation. Activated JAK dimer phosphorylates both the C-tails of receptor dimer and binding partners of a receptor (e.g. STAT family proteins). The literature data suggest that in parental MPN cells mainly JAK1 homodimers are formed, while in resistant cells mainly JAK2-JAK1 and JAK1-TYK2 hetero-dimers are formed. In other cases JAK-STAT hyper-activation can go through JAK1-JAK3 heterodimers, JAK1 homodimers, and other types of JAK dimers. Inventors' model demonstrates and experiments confirm that a combination of type I and type II JAK inhibitors can synergistically inhibit JAK kinase activity and suppress cellular proliferation in cells dependent on JAK-STAT pathway (FIGS. 16 and 17). Combining type I and type II inhibitors will allow the substantial decrease in the dose of a type II inhibitor without the decrease in efficiency of inhibition of the JAK-STAT pathway. Thus, in a situation when type I JAK inhibitors are ineffective in inhibition of JAK-STAT pathway, and type II JAK inhibitors have a narrow therapeutic window, it is a combination of type I and type II JAK inhibitors that can effectively inhibit JAK hetero-dimers using lower doses of combined drugs.

According to the inventors' model, a key thermodynamic factor that drives resistance to type I JAK inhibitors is the asymmetry factor (g1) for drug binding to a JAK homo- or hetero-dimer. Both homo- and hetero-dimers can become asymmetric after binding a single molecule of JAK inhibitor. In the model the asymmetry factor (g1) is 1 for symmetric dimers and is much greater than 1 for asymmetric homo- and hetero-dimers. Also, this factor depends on the type of inhibitor, and it is equal to 1 for type II inhibitors and greater than 1 for type I inhibitors. In this situation, although type II inhibitors alone can inhibit both JAK homo- and hetero-dimers, adding type I inhibitors results in synergistic effects in suppression of JAK dimers. Inhibition of symmetric homo-dimers is not significantly synergistic by combination of drugs, since both inhibitors are effective in such case (FIG. 16)

Inventors' model predictions were corroborated by cell line experiments where proliferation responses of T-ALL cells (DND41) to Type I inhibitor Ruxolitinib, Type II inhibitor CHZ868, and their combination were measured (FIG. 17). Inventors' results demonstrate synergy between different JAK inhibitors. However, the inventors observed saturation of inhibition of cellular proliferation. Even for doses exceeding 10 μM of each drug both alone and in combination, the inventors observed that plateau at the level of ˜60% suppression of cellular proliferation. This incomplete inhibition of proliferation is explained by activation of STAT family of transcription factors by alternative signalling pathways.

The forgoing embodiments are not intended to limit the scope of the protection afforded by the claims, but rather to describe examples of how the invention may be put into practice.

### REFERENCES

Adelmann, C. H., Ching, G., Du, L., Saporito, R. C., Bansal, V., Pence, L. J., Liang, R., Lee, W., and Tsai, K. Y. (2016). Comparative profiles of BRAF inhibitors: the paradox index as a predictor of clinical toxicity. Oncotarget 7, 30453-30460.
Arteaga, and Engelman. ERBB Receptors: From Oncogene Discovery to Basic Science to Mechanism-Based Cancer Therapeutics. Cancer Cell 25, 2014.
Baljuls, A., Kholodenko, B. N., and Kolch, W. (2013). It takes two to tango-signalling by dimeric Raf kinases. Mol Biosyst 9, 551-558.
Bastidas, A. C., Deal, M. S., Steichen, J. M., Keshwani, M. M., Guo, Y., and Taylor, S. S. (2012). Role of N-Terminal Myristylation in the Structure and Regulation of cAMP-Dependent Protein Kinase. Journal of molecular biology 422, 215-229.
Baxter, E. J. et al. Acquired mutation of the tyrosine kinase JAK2 in human myeloproliferative disorders. Lancet 365, 1054-1061 (2005).
Bessman, Nicholas J., Bagchi, A., Ferguson, Kathryn M., and Lemmon, Mark A. Complex Relationship between Ligand Binding and Dimerization in the Epidermal Growth Factor Receptor. Cell reports 9, 1306-1317.
Blinov, M. L., Faeder, J. R., Goldstein, B., and Hlavacek, W. S. (2004). BioNetGen: software for rule-based modeling of signal transduction based on the interactions of molecular domains. Bioinformatics.
Borisov, N. M., Chistopolsky, A. S., Faeder, J. R., and Kholodenko, B. N. (2008). Domain-oriented reduction of rule-based network models. IET Syst Biol 2, 342-351.
N., Weinstein, J. N., Collisson, E. A., Mills, G. B., Shaw, K. R., Ozenberger, B. A., Ellrott, K., Shmulevich, I., Sander, C., and Stuart, J. M. (2013). The Cancer Genome Atlas Pan-Cancer analysis project. Nat Genet 45, 1113-1120.
Caunt, C. J., Sale, M. J., Smith, P. D., and Cook, S. J. (2015). MEK1 and MEK2 inhibitors and cancer therapy: the long and winding road. Nature reviews Cancer 15, 577-592.
Chiloeches, A., Mason, C. S., and Marais, R. (2001). S338 phosphorylation of Raf-1 is independent of phosphatidylinositol 3-kinase and Pak3. Mol Cell Biol 21, 2423-2434.
Chou, T. C. (2006). Theoretical basis, experimental design, and computerized simulation of synergism and antagonism in drug combination studies. Pharmacol Rev 58, 621-681.
Chylek, L. A., Harris, L. A., Tung, C.-S., Faeder, J. R., Lopez, C. F., and Hlavacek, W. S. (2014). Rule-based modeling: a computational approach for studying biomolecular site dynamics in cell signaling systems. Wiley Interdisciplinary Reviews: Systems Biology and Medicine 6, 13-36.
Costello, J. C., Heiser, L. M., Georgii, E., Gonen, M., Menden, M. P., Wang, N. J., Bansal, M., Ammad-ud-din, M., Hintsanen, P., Khan, S. A., et al. (2014). A community effort to assess and improve drug sensitivity prediction algorithms. Nat Biotech 32, 1202-1212.
de Gramont, A., Watson, S., Ellis, L. M., Rodon, J., Tabernero, J., de Gramont, A., and Hamilton, S. R. (2015). Pragmatic issues in biomarker evaluation for targeted therapies in cancer. Nat Rev Clin Oncol 12, 197-212.
Dhillon, A. S., Meikle, S., Yazici, Z., Eulitz, M., and Kolch, W. (2002). Regulation of Raf-1 activation and signalling by dephosphorylation. EMBO J 21, 64-71.
Diaz, B., Barnard, D., Filson, A., MacDonald, S., King, A., and Marshall, M. (1997). Phosphorylation of Raf-1 serine 338-serine 339 is an essential regulatory event for Ras-dependent activation and biological signaling. Mol Cell Biol 17, 4509-4516.
Dougherty, M. K., Muller, J., Ritt, D. A., Zhou, M., Zhou, X. Z., Copeland, T. D., Conrads, T. P., Veenstra, T. D., Lu, K. P., and Morrison, D. K. (2005). Regulation of Raf-1 by direct feedback phosphorylation. Mol Cell 17, 215-224.
Endres, Engel, Das, Kovacs and Kuriyan. Regulation of the catalytic activity of the EGF receptor. Current Opinion in Structural Biology 2011, 21:777-784.
Fabbro, D. (2015). 25 years of small molecular weight kinase inhibitors: potentials and limitations. Mol Pharmacol 87, 766-775.
Faeder, J. R., Blinov, M. L., and Hlavacek, W. S. (2009). Rule-Based Modeling of Biochemical Systems with BioNetGen. In Systems Biology, I. V. Maly, ed. (Totowa, N. J.: Humana Press), pp. 113-167.
Fernandez-Fuentes, N., Madrid-Aliste, C. J., Rai, B. K., Fajardo, J. E., and Fiser, A. (2007a). M4T: a comparative protein structure modeling server. Nucleic Acids Research 35, W363-W368.
Fernandez-Fuentes, N., Rai, B. K., Madrid-Aliste, C. J., Eduardo Fajardo, J., and Fiser, A. (2007b). Comparative protein structure modeling by combining multiple templates and optimizing sequence-to-structure alignments. Bioinformatics 23, 2558-2565.
Forbes, S. A., Beare, D., Gunasekaran, P., Leung, K., Bindal, N., Boutselakis, H., Ding, M., Bamford, S., Cole, C., Ward, S., et al. (2015). COSMIC: exploring the world's knowledge of somatic mutations in human cancer. Nucleic Acids Res 43, D805-811.
Freeman, A. K., Ritt, D. A., and Morrison, D. K. (2013). Effects of Raf dimerization and its inhibition on normal and disease-associated Raf signaling. Mol Cell 49, 751-758.
Garnett, M. J., Rana, S., Paterson, H., Barford, D., and Marais, R. (2005). Wild-type and mutant B-RAF activate C-RAF through distinct mechanisms involving heterodimerization. Mol Cell 20, 963-969.
Garrett et al. Transcriptional and posttranslational up-regulation of HER3 (ErbB3) compensates for inhibition of the HER2 tyrosine kinase. PNAS, 2011, Vol. 108, No. 12, pp. 5021-5026.
Greco, W. R., Bravo, G., and Parsons, J. C. (1995). The search for synergy: a critical review from a response surface perspective. Pharmacol Rev 47, 331-385.
Grob, J. J., Amonkar, M. M., Karaszewska, B., Schachter, J., Dummer, R., Mackiewicz, A., Stroyakovskiy, D., Drucis, K., Grange, F., Chiarion-Sileni, V., et al. (2015). Comparison of dabrafenib and trametinib combination therapy with vemurafenib monotherapy on health-related quality of life in patients with unresectable or metastatic cutaneous BRAF Val600-mutation-positive melanoma (COMBI-v): results of a phase 3, open-label, randomised trial. The Lancet Oncology 16, 1389-1398.
Harris, L. A., Hogg, J. S., Tapia, J.-J., Sekar, J. A. P., Gupta, S., Korsunsky, I., Arora, A., Barua, D., Sheehan, R. P., and Faeder, J. R. (2016). BioNetGen 2.2: advances in rule-based modeling. Bioinformatics 32, 3366-3368.
Hatzivassiliou, G., Song, K., Yen, I., Brandhuber, B. J., Anderson, D. J., Alvarado, R., Ludlam, M. J., Stokoe, D., Gloor, S. L., Vigers, G., et al. (2010). RAF inhibitors prime wild-type RAF to activate the MAPK pathway and enhance growth. Nature 464, 431-435.
Heidorn, S. J., Milagre, C., Whittaker, S., Nourry, A., Niculescu-Duvas, I., Dhomen, N., Hussain, J., Reis-Filho, J. S., Springer, C. J., Pritchard, C., et al. (2010). Kinase-dead BRAF and oncogenic RAS cooperate to drive tumor progression through CRAF. Cell 140, 209-221.
Hindmarsh, A. C., Brown, P. N., Grant, K. E., Lee, S. L., Serban, R., Shumaker, D. E., and Woodward, C. S. (2005). SUNDIALS: Suite of nonlinear and differential/algebraic equation solvers. ACM Trans Math Softw 31, 363-396.
Holderfield, M., Deuker, M. M., McCormick, F., and McMahon, M. (2014). Targeting RAF kinases for cancer therapy: BRAF-mutated melanoma and beyond. Nature reviews Cancer 14, 455-467.
Hu, J., Stites, E. C., Yu, H., Germino, E. A., Meharena, H. S., Stork, P. J., Kornev, A. P., Taylor, S. S., and Shaw, A. S. (2013). Allosteric activation of functionally asymmetric RAF kinase dimers. Cell 154, 1036-1046.
Jambrina, P. G., Bohuszewicz, O., Buchete, N. V., Kolch, W., and Rosta, E. (2014). Molecular mechanisms of asymmetric RAF dimer activation. Biochem Soc Trans 42, 784-790.
Jambrina, P. G., Rauch, N., Pilkington, R., Rybakova, K., Nguyen, L. K., Kholodenko, B. N., Buchete, N. V., Kolch, W., and Rosta, E. (2016). Phosphorylation of RAF Kinase Dimers Drives Conformational Changes that Facilitate Transactivation. Angew Chem Int Ed Engl 55, 983-986.
James, C. et al. A unique clonal JAK2 mutation leading to constitutive signalling causes polycythaemia vera. Nature 434, 1144-1148 (2005).
Johnson, D. B., Menzies, A. M., Zimmer, L., Eroglu, Z., Ye, F., Zhao, S., Rizos, H., Sucker, A., Scolyer, R. A., Gutzmer, R., et al. (2015). Acquired BRAF inhibitor resistance: A multicenter meta-analysis of the spectrum and frequencies, clinical behaviour, and phenotypic associations of resistance mechanisms. European Journal of Cancer 51, 2792-2799.
Jura N., Zhang X., Endres N. F., Seeliger M. A., Schindler T., Kuriyan J. Catalytic Control in the EGF Receptor and Its Connection to General Kinase Regulatory Mechanisms. Molecular Cell 42, pp. 9-22, 2011.
Karoulia, Z., Wu, Y., Ahmed, T. A., Xin, Q., Bollard, J., Krepler, C., Wu, X., Zhang, C., Bollag, G., Herlyn, M., et al. (2016). An Integrated Model of RAF Inhibitor Action Predicts Inhibitor Activity against Oncogenic BRAF Signaling. Cancer Cell 30, 485-498.
Keith, C. T., Borisy, A. A., and Stockwell, B. R. (2005). Multicomponent therapeutics for networked systems. Nat Rev Drug Discov 4, 71-78.
Kholodenko, B., Zilinskiene, V., Borutaite, V., Ivanoviene, L., Toleikis, A., and Praskevicius, A. (1987). The role of adenine nucleotide translocators in regulation of oxidative phosphorylation in heart mitochondria. FEBS Lett 223, 247-250.
Kholodenko, B. N. (2015). Drug resistance resulting from kinase dimerization is rationalized by thermodynamic factors describing allosteric inhibitor effects. Cell reports 12, 1939-1949.
Kholodenko, B. N., and Birtwistle, M. R. (2009). Four-dimensional dynamics of MAPK information processing systems. Wiley Interdiscip Rev Syst Biol Med 1, 28-44.
Kholodenko, B. N., Demin, O. V., and Westerhoff, H. V. (1997). Control Analysis of Periodic Phenomena in Biological Systems. The Journal of Physical Chemistry B 101, 2070-2081
Kholodenko, B. N., Hoek, J. B., and Westerhoff, H. V. (2000). Why cytoplasmic signalling proteins should be recruited to cell membranes. Trends Cell Biol 10, 173-178.
Kholodenko, B. N., and Westerhoff, H. V. (1995). The macroworld versus the microworld of biochemical regulation and control. Trends in Biochemical Sciences 20, 52-54.
King, A. J., Patrick, D. R., Batorsky, R. S., Ho, M. L., Do, H. T., Zhang, S. Y., Kumar, R., Rusnak, D. W., Takle, A. K., Wilson, D. M., et al. (2006). Demonstration of a Genetic Therapeutic Index for Tumors Expressing Oncogenic &lt;em&gt;BRAF&lt;/em&gt; by the Kinase Inhibitor SB-590885. Cancer research 66, 11100.
Kleiman, L. B., Maiwald, T., Conzelmann, H., Lauffenburger, D. A., and Sorger, P. K. (2011). Rapid phospho-turnover by receptor tyrosine kinases impacts downstream signaling and drug binding. Mol Cell 43, 723-737.
Kralovics, R. et al. A gain-of-function mutation of JAK2 in myeloproliferative disorders. N. Engl. J. Med. 352, 1779-1790 (2005)
Klein P, Mattoon D, Lemmon M A and Schlessinger J. A structure-based model for ligand binding and dimerization of EGF receptors. PNAS Jan. 27, 2004. 101 (4) 929-934.
Koppikar, P. et al. Heterodimeric JAK-STAT activation as a mechanism of persistence to JAK2 inhibitor therapy. Nature 489, 155-160 (2012).
Larkin, J., Ascierto, P. A., Dreno, B., Atkinson, V., Liszkay, G., Maio, M., Mandala, M., Demidov, L., Stroyakovskiy, D., Thomas, L., et al. (2014). Combined vemurafenib and cobimetinib in BRAF-mutated melanoma. The New England journal of medicine 371, 1867-1876.
Lavoie, H., and Therrien, M. (2015). Regulation of RAF protein kinases in ERK signalling. Nat Rev Mol Cell Biol 16, 281-298.
Lito, P., Rosen, N., and Solit, D. B. (2013). Tumor adaptation and resistance to RAF inhibitors. Nat Med 19, 1401-1409.
Macdonald-Obermann J. L., Adak S., Landgraf R., Piwnica-Worms D., Pike L. J. Dynamic Analysis of the Epidermal Growth Factor (EGF) Receptor-ErbB2-ErbB3 Protein Network by Luciferase Fragment Complementation Imaging. The Journal of Biological Chemistry Vol. 288, No. 42, pp. 30773-30784, 2013.
MacKerell, A. D., Bashford, D., Bellott, M., Dunbrack, R. L., Evanseck, J. D., Field, M. J., Fischer, S., Gao, J., Guo, H., Ha, S., et al. (1998). All-Atom Empirical Potential for Molecular Modeling and Dynamics Studies of Proteins. The Journal of Physical Chemistry B 102, 3586-3616.
Mackerell, A. D., Feig, M., and Brooks, C. L. (2004). Extending the treatment of backbone energetics in protein force fields: Limitations of gas-phase quantum mechanics in reproducing protein conformational distributions in molecular dynamics simulations. Journal of Computational Chemistry 25, 1400-1415.
Nan, X., Tamguney, T. M., Collisson, E. A., Lin, L. J., Pitt, C., Galeas, J., Lewis, S., Gray, J. W., McCormick, F., and Chu, S. (2015). Ras-GTP dimers activate the Mitogen-Activated Protein Kinase (MAPK) pathway. Proc Natl Acad Sci USA 112, 7996-8001.
Nazarian, R., Shi, H., Wang, Q., Kong, X., Koya, R. C., Lee, H., Chen, Z., Lee, M. K., Attar, N., Sazegar, H., et al. (2010). Melanomas acquire resistance to B-RAF(V600E) inhibition by RTK or N-RAS upregulation. Nature 468, 973-977.
Niyazi, M., Niyazi, I., and Belka, C. (2007). Counting colonies of clonogenic assays by using densitometric software. Radiation Oncology 2, 4.
Nussinov, R., Tsai, C.-J., and Ma, B. (2013). The Underappreciated Role of Allostery in the Cellular Network. Annual Review of Biophysics 42, 169-189.
Okaniwa, M., Hirose, M., Arita, T., Yabuki, M., Nakamura, A., Takagi, T., Kawamoto, T., Uchiyama, N., Sumita, A., Tsutsumi, S., et al. (2013). Discovery of a selective kinase inhibitor (TAK-632) targeting pan-RAF inhibition: design, synthesis, and biological evaluation of C-7-substituted 1,3-benzothiazole derivatives. J Med Chem 56, 6478-6494.
Pardanani, A. et al. Safety and efficacy of TG101348, a selective JAK2 inhibitor, in myelofibrosis. J. Clin. Oncol. 29, 789-796 (2011).
Pawson, T., and Nash, P. (2003). Assembly of cell regulatory systems through protein interaction domains Science 300, 445-452.
Pikman, Y. et al. MPLW515L is a novel somatic activating mutation in myelo fibrosis with myeloid metaplasia. PLoS Med. 3, e270 (2006).
Poulikakos, P. I., Persaud, Y., Janakiraman, M., Kong, X., Ng, C., Moriceau, G., Shi, H., Atefi, M., Titz, B., Gabay, M. T., et al. (2011). RAF inhibitor resistance is mediated by dimerization of aberrantly spliced BRAF(V600E). Nature 480, 387-390.
Poulikakos, P. I., Zhang, C., Bollag, G., Shokat, K. M., and Rosen, N. (2010). RAF inhibitors transactivate RAF dimers and ERK signalling in cells with wild-type BRAF. Nature 464, 427-430.
Poussin, C., Mathis, C., Alexopoulos, L. G., Messinis, D. E., Dulize, R. H. J., Belcastro, V., Melas, I. N., Sakellaropoulos, T., Rhrissorrakrai, K., Bilal, E., et al. (2014). The species translation challenge—A systems biology perspective on human and rat bronchial epithelial cells. 1, 140009.
Prasad, V. (2016). Perspective: The precision-oncology illusion. Nature 537, S63-S63. Prior, I. A., Lewis, P. D., and Mattos, C. (2012). A comprehensive survey of Ras mutations in cancer. Cancer research 72, 2457-2467.
Qin, J., Xie, P., Ventocilla, C., Zhou, G., Vultur, A., Chen, Q., Liu, Q., Herlyn, M., Winkler, J., and Marmorstein, R. (2012). Identification of a Novel Family of BRAFV600E Inhibitors. Journal of Medicinal Chemistry 55, 5220-5230.
Rahman, M. A., Salajegheh, A., Smith, R. A., and Lam, A. K. (2014). BRAF inhibitors: From the laboratory to clinical trials. Crit Rev Oncol Hematol 90, 220-232.
Rauch, N., Rukhlenko, O. S., Kolch, W., and Kholodenko, B. N. (2016). MAPK kinase signalling dynamics regulate cell fate decisions and drug resistance. Current Opinion in Structural Biology 41, 151-158.
Ritt, D. A., Monson, D. M., Specht, S. I., and Morrison, D. K. (2010). Impact of feedback phosphorylation and Raf heterodimerization on normal and mutant B-Raf signaling. Mol Cell Biol 30, 806-819.
Romano, D., Nguyen, L. K., Matallanas, D., Halasz, M., Doherty, C., Kholodenko, B. N., and Kolch, W. (2014). Protein interaction switches coordinate Raf-1 and MST2/Hippo signalling. Nat Cell Biol 16, 673-684.
Roskoski, R., Jr. (2016). Classification of small molecule protein kinase inhibitors based upon the structures of their drug-enzyme complexes. Pharmacol Res 103, 26-48.
Rubinstein, B. Y., Mattingly, H. H., Berezhkovskii, A. M., and Shvartsman, S. Y. (2016). Long-term dynamics of multisite phosphorylation. Molecular Biology of the Cell 27, 2331-2340.
Rushworth, L. K., Hindley, A. D., O'Neill, E., and Kolch, W. (2006). Regulation and role of Raf-1/B-Raf heterodimerization. Mol Cell Biol 26, 2262-2272.
Ryckaert, J.-P., Ciccotti, G., and Berendsen, H. J. C. (1977). Numerical integration of the cartesian equations of motion of a system with constraints: molecular dynamics of n-alkanes. Journal of Computational Physics 23, 327-341.
Rykunov, D., Steinberger, E., Madrid-Aliste, C. J., and Fiser, A. (2009). Improved scoring function for comparative modeling using the M4T method. Journal of Structural and Functional Genomics 10, 95-99.
Saez-Rodriguez, J., MacNamara, A., and Cook, S. (2015). Modeling Signaling Networks to Advance New Cancer Therapies. Annu Rev Biomed Eng 17, 143-163.
Sanchez-Laorden, B., Viros, A., Girotti, M. R., Pedersen, M., Saturno, G., Zambon, A., Niculescu-Duvaz, D., Turajlic, S., Hayes, A., Gore, M., et al. (2014). BRAF Inhibitors Induce Metastasis in RAS Mutant or Inhibitor-Resistant Melanoma Cells by Reactivating MEK and ERK Signaling. Science Signaling 7, ra30.
M Scaltriti, C Verma, M Guzman, J Jimenez, J L Parra, K Pedersen, D J Smith, S Landolfi, S Ramon y Cajal, J Arribas and J Baselga. Lapatinib, a HER2 tyrosine kinase inhibitor, induces stabilization and accumulation of HER2 and potentiates trastuzumab-dependent cell cytotoxicity. Oncogene (2009) 28, pp. 803-814.
Shao, Q., Xu, Z., Wang, J., Shi, J., and Zhu, W. (2017). Energetics and structural characterization of the “DFG-flip” conformational transition of B-RAF kinase: a SITS molecular dynamics study. Phys Chem Chem Phys 19, 1257-1267.
Shi, H., Moriceau, G., Kong, X., Lee, M. K., Lee, H., Koya, R. C., Ng, C., Chodon, T., Scolyer, R. A., Dahlman, K. B., et al. (2012). Melanoma whole-exome sequencing identifies (V600E)B-RAF amplification-mediated acquired B-RAF inhibitor resistance. Nat Commun 3, 724.
Stephen, Andrew G., Esposito, D., Bagni, Rachel K., and McCormick, F. Dragging Ras Back in the Ring. Cancer Cell 25, 272-281.
Sturm, O. E., Orton, R., Grindlay, J., Birtwistle, M., Vyshemirsky, V., Gilbert, D., Calder, M., Pitt, A., Kholodenko, B., and Kolch, W. (2010). The mammalian MAPK/ERK pathway exhibits properties of a negative feedback amplifier. Sci Signal 3, ra90.
Varga, A., Ehrenreiter, K., Aschenbrenner, B., Kocieniewski, P., Kochanczyk, M., Lipniacki, T., and Baccarini, M. (2017). RAF1/BRAF dimerization integrates the signal from RAS to ERK and ROKalpha. Sci Signal 10.
Verstovsek, S. et al. Safety and efficacy of INCB018424, a JAK1 and JAK2 inhibitor, in myelofibrosis. N. Engl. J. Med. 363, 1117-1127 (2010).
Wan, P. T. C., Garnett, M. J., Roe, S. M., Lee, S., Niculescu-Duvaz, D., Good, V. M., Project, C. G., Jones, C. M., Marshall, C. J., Springer, C. J., et al. (2004). Mechanism of Activation of the RAF-ERK Signaling Pathway by Oncogenic Mutations of B-RAF. Cell 116, 855-867.
Weber, C. K., Slupsky, J. R., Kalmes, H. A., and Rapp, U. R. (2001). Active Ras Induces Heterodimerization of cRaf and BRaf. Cancer research 61, 3595.
Wenglowsky, S., Ren, L., Ahrendt, K. A., Laird, E. R., Aliagas, I., Alicke, B., Buckmelter, A. J., Choo, E. F., Dinkel, V., Feng, B., et al. (2011). Pyrazolopyridine Inhibitors of B-RafV600E. Part 1: The Development of Selective, Orally Bioavailable, and Efficacious Inhibitors. ACS Medicinal Chemistry Letters 2, 342-347.
Wilhelm, S. M., Carter, C., Tang, L., Wilkie, D., McNabola, A., Rong, H., Chen, C., Zhang, X., Vincent, P., McHugh, M., et al. (2004). BAY 43-9006 Exhibits Broad Spectrum Oral Antitumor Activity and Targets the RAF/MEK/ERK Pathway and Receptor Tyrosine Kinases Involved in Tumor Progression and Angiogenesis. Cancer research 64, 7099.
Yaktapour, N., Meiss, F., Mastroianni, J., Zenz, T., Andrlova, H., Mathew, N. R., Claus, R., Hutter, B., Frohling, S., Brors, B., et al. (2014). BRAF inhibitor-associated ERK activation drives development of chronic lymphocytic leukemia. J Clin Invest 124, 5074-5084.
Yao, Z., Torres, N. M., Tao, A., Gao, Y., Luo, L., Li, Q., de Stanchina, E., Abdel-Wahab, O., Solit, D. B., Poulikakos, P. I., et al. (2015). BRAF Mutants Evade ERK-Dependent Feedback by Different Mechanisms that Determine Their Sensitivity to Pharmacologic Inhibition. Cancer Cell 28, 370-383.
Yeh, P. J., Hegreness, M. J., Aiden, A. P., and Kishony, R. (2009). Drug interactions and the evolution of antibiotic resistance. Nat Rev Microbiol 7, 460-466.
Zehir, A., Benayed, R., Shah, R. H., Syed, A., Middha, S., Kim, H. R., Srinivasan, P., Gao, J., Chakravarty, D., Devlin, S. M., et al. (2017). Mutational landscape of metastatic cancer revealed from prospective clinical sequencing of 10,000 patients. Nat Med 23, 703-713.
Zhang, C., Spevak, W., Zhang, Y., Burton, E. A., Ma, Y., Habets, G., Zhang, J., Lin, J., Ewing, T., Matusow, B., et al. (2015). RAF inhibitors that evade paradoxical MAPK pathway activation. Nature 526, 583-586.
Zhao, R. et al. Identification of an acquired JAK2 mutation in polycythemia vera. J. Biol. Chem. 280, 22788-22792 (2005).

1. A composition for use in the prevention, management, amelioration or treatment of a cancer, the composition comprising two enzyme inhibitors targeting two different conformations of an enzyme, or enzymes in the same functional family, which has, or have, been implicated in the cancer.

2. The composition as claimed in claim 1, wherein enzyme activation including kinase dimerization or oligomerization is a component of the onset or progress of the cancer and the targeting of said enzymes causes inhibition of the enzyme dimers or oligomers.

3. The composition as claimed in either claim 1 or 2, wherein the two synergistic enzyme inhibitors act synergistically.

4. A composition for use in the prevention, management, amelioration or treatment of a RASopathy disorder, the composition comprising two enzyme inhibitors targeting two different conformations of an enzyme, or enzymes in the same functional family, which has, or have, been implicated in the RASopathy disorder.

5. The composition as claimed in any preceding claim, wherein the enzyme, or enzymes, comprise a kinase or pseudokinase, and the two enzyme inhibitors comprise two kinase or pseudokinase inhibitors.

6. The composition as claimed in claim 5, wherein the two kinase or pseudokinase inhibitors target alternative conformations of the DFG motif and/or αC-helix on the kinase.

7. The composition as claimed in either claim 5 or 6, wherein the two kinase inhibitors are of different types.

8. The composition as claimed in claim 7, wherein the different types comprise Type I and Type II or Type I 1/2 and Type II kinase inhibitors.

9. The composition as claimed in any one of claims 5 to 7, wherein at least one of the kinase inhibitors comprises a covalent inhibitor.

10. The composition as claimed in any preceding claim, wherein the two enzyme inhibitors are RAF inhibitors.

11. The composition as claimed in any preceding claim, wherein the composition targets activating mutations and/or overexpressed proteins in one or more of the following: RAS, RAF, ErbB and JAK family proteins.

12. The composition as claimed in claim 11, wherein the mutation comprises a BRAF mutant comprising the BRAFV600E mutation.

13. The composition as claimed in claim 11, wherein the mutation comprises a RAS (H-RAS, N-RAS or K-RAS) mutant and the inhibitors comprise CI/DO (DFG-OUT, αC-IN, Type II) and CO/DI (DFG-IN, αC-OUT, Type I1/2) inhibitors.

14. The composition as claimed in any one of claims 1 to 9, wherein the two enzyme inhibitors comprise two ErbB family kinase inhibitors.

15. The composition as claimed in any one of claims 1 to 9, wherein the two enzyme inhibitors comprise two JAK family kinase inhibitors.

16. The composition as claimed in either claim 13 or claim 14, wherein the ErbB inhibitor is specific to one or more of the following proteins: Her1 (EGFR, ErbB1), Her2 (Neu, ErbB2), Her3 (ErbB3), and Her4 (ErbB4); and the JAK inhibitor is specific to one or more of the following proteins: JAK1, JAK2, JAK3, and TYK2.

17. The composition as claimed in any preceding claim, wherein the each enzyme inhibitor is present in the composition at a lower dose than would typically be used individually for treatment.

18. A combination therapy for use in the prevention, management, amelioration or treatment of a cancer, the combination comprising two enzyme inhibitors targeting two different conformations of an enzyme, or enzymes in the same functional family, which has, or have, been implicated in the cancer.

19. The combination as claimed in claim 18, wherein enzyme activation including kinase dimerization or oligomerization is a component of the onset or progress of the cancer and the targeting of said enzymes causes inhibition of the enzyme dimers or oligomers.

20. The combination as claimed in either claim 18 or 19, wherein the two synergistic enzyme inhibitors act synergistically.

21. A combination therapy for use in the prevention, management, amelioration or treatment of a RASopathy disorder, in which targeted therapy is used, the combination comprising two enzyme inhibitors targeting two different conformations of an enzyme, or enzymes in the same functional family, which has, or have, been implicated in the RASopathy disorder.

22. The combination as claimed in any of claim 18 or 21, wherein the enzyme comprises a kinase, or kinases, a pseudokinase or pseudokinases, and the two enzyme inhibitors comprise two kinase inhibitors.

23. The combination as claimed in claim 22, wherein the two kinase inhibitors target alternative conformations of the DFG motif and/or αC-helix on the kinase or pseudokinase.

24. The combination as claimed in either claim 22 or 23, wherein the two kinase inhibitors are of different types.

25. The combination as claimed in claim 24, wherein the different types comprise Type I and Type II or Type I 1/2 and Type II kinase inhibitors.

26. The combination as claimed in any one of claims 22 to 25, wherein at least one of the kinase inhibitors comprises a covalent inhibitor.

27. The combination as claimed in any one of claims 18 to 25, wherein the two enzyme inhibitors are RAF inhibitors.

28. The combination as claimed in any one of claims 18 to 27, wherein the mutation comprises activating mutations and/or overexpressed proteins in RAS and/or RAF family proteins; ErbB family proteins; and JAK family proteins.

29. The combination as claimed in claim 28, wherein the mutation comprises a BRAF mutant comprising BRAFV600E mutation.

30. The combination as claimed in claim 28, wherein the mutation comprises a RAS mutant and the inhibitors comprise CI/DO (DFG-OUT, αC-IN, Type II) and CO/DI (DFG-IN, αC-OUT, Type I1/2) inhibitors.

31. The composition as claimed in any one of claims 18 to 26, wherein the two enzyme inhibitors comprise two ErbB family kinase inhibitors.

32. The composition as claimed in any one of claims 18 to 26, wherein the two enzyme inhibitors comprise two JAK family kinase inhibitors.

33. The composition as claimed in claim 31, wherein the ErbB inhibitor is specific to one or more of the following proteins: Her1 (EGFR, ErbB1), Her2 (Neu, ErbB2), Her3 (ErbB3), and Her4 (ErbB4).

34. The composition as claimed in claim 32 wherein the JAK inhibitor is specific to one or more of the following proteins: JAK1, JAK2, JAK3, and TYK2.

35. The combination as claimed in any one of claims 17 to 31, wherein the each enzyme inhibitor is present in the composition at a lower dose than would typically be used for treatment.

36. The composition as claimed in any one of claims 1 to 8, 10 to 14 and 17, or the combination as claimed in any one of claims 18 to 25, 27 to 30 and 35, wherein the each enzyme inhibitor is selected from one the following groups:
(i) any Type I1/2 RAF inhibitor (e.g. vemurafenib, dabrafenib, LGX818, PLX8394); and
(ii) any Type II RAF inhibitor (e.g. sorafenib, AZ-628, TAK-632, LY3009120, BGB283)

37. The composition as claimed in any one of claims 1 to 8, 10 to 14 and 17, or the combination as claimed in any one of claims 18 to 25, 27 to 30 and 35, wherein the each enzyme inhibitor is selected from one the following groups:
(i) any Type I RAF inhibitor (e.g. SB-590885, GDC-0879); and
(ii) any Type II RAF inhibitor (e.g. sorafenib, AZ-628, TAK-632, LY3009120, BGB283)

38. The composition as claimed in any one of claims 1 to 8, 14, 16, 17, or the combination as claimed in any one of claims 18 to 25, 31, 33, 35, wherein the each enzyme inhibitor is selected from one the following groups:
(i) any Type I ErbB inhibitor (e.g. gefitinib, erlotinib); and
(ii) any Type I1/2 ErbB inhibitor (e.g. lapatinib)

39. The composition as claimed in any one of claims 1 to 8, 14, 16, 17, or the combination as claimed in any one of claims 18 to 25, 32, 34, 35, wherein the each enzyme inhibitor is selected from one the following groups:
(i) any Type I JAK inhibitor (e.g. tofacitinib, ruxolitinib); and
(ii) any Type II JAK inhibitor (e.g. BBT-594, CHZ868)

40. A method of identifying two or more compounds for incorporation into a combination therapy for the use in the treatment of cancer or a RASopathy disorder, where enzyme activation including kinase dimerization or oligomerization is a component of the onset or progress of the cancer or RASopathy disorder, the method comprising the steps of:
a) identifying if the cancer or RASopathy disorder involves enzyme activation which includes homodimerization or heterodimerization of the enzyme and/or within the same enzyme family; and
b) identifying if enzyme inhibitors are capable of changing allosteric interactions of enzyme protomers in a dimer; and
c) selecting two inhibitors targeting different conformations of the same enzyme, or enzymes in the same enzyme family, and including the inhibitors in a combination therapy.

41. A method of formulating a composition for use in the prevention, management, amelioration or treatment of a cancer or RASopathy disorder, the composition comprising combining two enzyme inhibitors capable of targeting two different conformations of an enzyme, or enzymes in the same functional family, implicated in the cancer or RASopathy disorder.

42. The method as claimed in claim 41, wherein enzyme activation including kinase dimerization or oligomerization is a component of the onset or progress of the cancer or RASopathy disorder and the targeting of said enzymes causes inhibition of the enzyme dimers or oligomers.

43. The method as claimed in either claim 41 or 42, wherein the two synergistic enzyme inhibitors act synergistically.

44. The method of any of claims 41 to 43, wherein the method is used to produce a composition according to any one of claims 1 to 17 or a combination therapy according to any one of claims 18 to 35.

45. The method of claim 44, wherein the disease involves enzyme activation including kinase or kinase-pseudokinase dimerization or oligomerization.

46. The method of claim 45, wherein the enzyme activation includes homodimerization or heterodimerization with the enzyme, or within the same enzyme family.

47. The method of claim 46, wherein the inhibitors are capable of changing allosteric interactions of enzyme protomers in a dimer or oligomer.
